(19) United States
(12) Patent Application Publication
(10) Pub. No.: US 2013/0337500 A1
(43) Pub. Date: Dec. 19, 2013

(54) SYSTEM AND METHOD FOR ISOLATION OF CELLS

(71) Applicants: Swee Jin Tan, Singapore (SG); Chwee Teck Lim, Singapore (SG)

(72) Inventors: Swee Jin Tan, Singapore (SG); Chwee Teck Lim, Singapore (SG)

(73) Assignee: National University of Singapore, Singapore (SG)

(21) Appl. No.: 12/765,576

(22) Filed: Apr. 22, 2010

Related U.S. Application Data
(60) Provisional application No. 61/172,250, filed on Apr. 24, 2009.

(51) Int. Cl. C12Q 1/06; C12N 5/078; C12Q 1/04; C12M 1/00

(52) U.S. Cl. 435/39; 435/308.1; 435/325; 435/34

(57) ABSTRACT

In accordance with an embodiment of the invention, there is provided a microfluidic device for isolating cells from a biological fluid. The device comprises an inlet receiving the biological fluid flowed into the device, and at least one array of a plurality of isolation wells receiving the biological fluid from the inlet. At least one isolation well of the plurality of isolation wells comprises a cell trap of a size and shape suitable to mechanically isolate a cell within the cell trap. The cell trap comprises at least one gap of a size and shape suitable to prevent passage of the cells to be isolated but to permit passage of other components of the biological fluid through the cell trap.

### RELATED APPLICATION

This application claims the benefit of U.S. Provisional Application No. 61/172,250, filed on Apr. 24, 2009, the entire teachings of the which application are incorporated herein by reference.

### BACKGROUND OF THE INVENTION

Cancer is a leading cause of death globally and early detection is one of the most effective means to combat the disease. Recent clinical studies show that the number of cancer cells in cancer patients' blood can predict the disease development and treatment efficacy. Studying these cells may also lead to a better understanding of the disease. Moreover, getting access to blood samples is relatively easy and less invasive and painful than tumor biopsies.

Isolation and enumeration of circulating tumor cells (CTCs) in peripheral blood has clinical significance in combating cancer (J. M. Reuben, S. Krishnamurthy, W. Woodward, M. Cristofanilli, Expert Opin. Med. Diagnostics 2, 339 (2008); S. Urtishak, R. K. Alpaugh, L. M. Weiner, R. F. Swaby, Biomarkers Med. 2, 137 (2008)). Deaths resulting from cancer are mainly due to late diagnosis of the disease and when metastasis has occurred (G. P. Gupta, J. Massague, Cell 127, 679-695 (2006); P. S. Steeg. Nat. Med. 12, 895 (2006)). To ensure patients receive timely treatment, enumerating CTCs in blood can complement existing early detection methods. Furthermore, blood samples being a routinely extracted body fluid in any health test can be easily attained to check for CTCs. CTCs are found in patients with metastatic carcinomas (W. J. Allard, J. Matera, M. C. Miller, M. Repollet, M. C. Connelly, C. Rao, A. G. Tibbe, J. W. Uhr, L. W. Terstappen, Clin. Cancer Res. 10, 6897 (2004); S. Steen, J. Nemunaitis, T. Fisher, J. Kuhn. Proc 21, 127 (2008), Bayl Univ Med. Cent.) and are associated with the disease progression (M. Cristofanilli, G. T. Budd, M. J. Ellis, A. Stopeck, J. Matera, M. C. Miller, J. M. Reuben, G. V. Doyle, W. J. Allard, L. W. Terstappen, D. F. Hayes, N. Engl. J. Med. 351, 781 (2004); S. Mocellin, D. Hoon, A. Ambrosi, D. Nitti, C. R. Rossi, Clin. Cancer Res. 12, 4605 (2006); G. Wiedswang, B. Naume, Nat. Clin. Pract. Oncol. 4, 154 (2007)). The effectiveness of therapeutic treatments can also be measured by the number of CTCs in blood (F. Nole, E. Munzone, L. Zorzino, I. Minchella, M. Salvatici, E. Botteri, M. Medici, E. Verri, L. Adamoli, N. Rotmensz, A. Goldhirsch, M. T. Sandri, Ann. Oncol. 19, 891 (2008); A. Rolle, R. Gunzel, U. Pachmann, B. Willen, K. Hoffken, K. Pachmann, World J. Surg. Oncol. 3, 18 (2005)). Thus, there is much interest in isolating, quantifying and studying these cells obtained from peripheral blood.

CTCs are of very low concentration in blood which poses the technical difficulty to detect these rare cells (J. E. Losanoff, W. Zhu, W. Qin, F. Mannello, E. R. Sauter, Breast 17, 540 (2008); V. Zieglschmid, C. Hollmann, O. Bocher, Crit. Rev. Clin. Lab. Sci. 42, 155 (2005)). The absolute number of CTCs in blood of cancer patients varies and depends on the conditions of the patients. Leading techniques to enumerate CTCs include immuno-magnetic separation followed by immunocytochemistry detection, such as the CellSearch® system sold by Veridex LLC (a Johnson & Johnson company) of Raritan, N.J., U.S.A. (M. Cristofanilli, G. T. Budd, M. J. Ellis, A. Stopeck, J. Matera, M. C. Miller, J. M. Reuben, G. V. Doyle, W. J. Allard, L. W. Terstappen, D. F. Hayes, N. Engl. J. Med. 351, 781 (2004); H. Yagata, S, Nakamura, M. Toi, H. Bando, S. Ohno, A. Kataoka, Int J Clin Oncol 13, 252 (2008)). A further leading technique is RT-PCR to indicate the presence of CTCs in peripheral blood (L. A. Mattano Jr., T. J. Moss, S. G. Emerson, Cancer Res. 52, 4701 (1992); C. P. Schroder, M. H. Ruiters, S. de Jong, A. T. Tiebosch, J. Wesseling, R. Veenstra, J. de Vries, H. J. Hoekstra, L. F. de Leij, E. G. de Vries, Int. J. Cancer 106, 611 (2003)). These methods have been successfully demonstrated on various cancer types (S. Dawood, K. Broglio, V. Valero, J. Reuben, B. Handy, R. Islam, S. Jackson, G. N. Hortobagyi, H. Fritsche, M. Cristofanilli, Cancer 113, 2422 (2008); R. Szatanek, G. Drabik, J. Baran, P. Kolodziejczyk, J. Kulig, J. Stachura, M. Zembala, Oncol. Rep. 19, 1055 (2008); C. S. Wong, M. T. Cheung, B. B. Ma, E. Pun Hui, A. C. Chan, C. K. Chan, K. C. Lee, W. Cheuk, M. Y. Lam, M. C. Wong, C. M. Chan, J. K. Chan, and A. T. Chan, Int. J. Surg. Pathol. 16, (2008)). Alternative methodologies such as a direct visualization assay (H. J. Kahn, A. Presta, L. Y. Yang, J. Blondal, M. Trudeau, L. Lickley, C. Holloway, D. R. McCready, D. Maclean, A. Marks, Breast Cancer Res. Treat. 86, 237 (2004)), fluorescent activated cell sorter (FACS) (J. G. Moreno, S. M. O'Hara, S. Gross, G. Doyle, H. Fritsche, L. G. Gomella, L. W. Terstappen, Urology 58, 386 (2001)), fibre-optic array scanning technology (FAST) cytometer (R. T. Krivacic, A. Ladanyi, D. N. Curry, H. B. Hsieh, P. Kuhn, D. E. Bergsrud, J. F. Kepros, T. Barbera, M. Y. Ho, L. B. Chen, R. A. Lerner, R. H. Bruce, Proc. Natl. Acad. Sci. USA 101, 10501 (2004)) and anti-EpCAM coated microfabricated structures (S. Nagrath, L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, M. Toner, Nature 450, 1235 (2007)) have also been used to enumerate CTCs in blood samples.

Complex procedures, tedious inspections and long processing time are the limiting factors associated with most existing techniques. Furthermore, viability of the isolated cells are lost as fixing of the samples is required by most existing techniques. There is much to understand about the condition of CTCs whilst in circulation (K. Pantel. R. H. Brakenhoff, B. Brandt, Nat. Rev. Cancer 8, 329 (2008)) and having viable cells after isolation would allow studies to be carried out on CTC sub-populations. This may provide valuable insights to the biological characteristics of the disease such as the link between cancer stem cells and metastasis. Although a recent study isolated CTCs (S. Nagrath, L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, M. Toner, Nature 450, 1235 (2007)), the isolated cells are likely difficult to retrieve due to the binding of the tumor associated antigens to the device. Retrieving these cells may require high mechanical forces or biochemical agents and the integrity of these cells might be affected as a result (S. F. Chang, C. A. Chang, D. Y. Lee, P. L. Lee, Y. M. Yeh, C. R. Yeh, C. K. Cheng, S. Chien, J. J. Chiu, Proc. Natl. Acad. Sci. USA 105, 3927 (2008)). In addition, most methodologies will require functional modifications which is less desirable (0. Lara, X. Tong, M. Zborowski, J. J. Chalmers, Exp. Hematol. 32, 891 (2004)).

### SUMMARY OF THE INVENTION

In accordance with an embodiment of the invention, there is provided a microfluidic device for isolating cells from a biological fluid. The device comprises an inlet receiving the biological fluid flowed into the device; and at least one array of a plurality of isolation wells receiving the biological fluid from the inlet, at least one isolation well of the plurality of isolation wells comprising a cell trap of a size and shape suitable to mechanically isolate a cell within the cell trap, the cell trap comprising at least one gap of a size and shape suitable to prevent passage of the cells to be isolated but to permit passage of other components of the biological fluid through the cell trap.

In further, related embodiments, the biological fluid may comprise blood, the cells to be isolated may comprise circulating tumor cells, and the other components may comprise blood cells. The device may further comprise a cell collection point receiving isolated circulating tumor cells from the array of isolation wells; and a waste outlet receiving waste blood cells from the isolation wells. The cell trap may comprise a crescent-shaped structure. The cell trap may comprise at least one of a “U” shaped structure, a “V” shaped structure and/or a “C” shaped structure. The at least one array of the plurality of isolation wells may comprise a plurality of rows of isolation wells, which may be offset from each other, for example by about 25 μm. The at least one array of the plurality of isolation wells may comprise at least one row of isolation wells, the isolation wells of the at least one row being spaced apart from each other by a distance sufficient to prevent clogging of the at least one array by blood cells, for example by about 50 μm. A pressure differential between the inlet and a waste outlet of the device may be within the range of physiological pressure differences found in circulating whole blood.

In further related embodiments, the device may further comprise a pre-filter receiving the biological fluid from the inlet and flowing pre-filtered biological fluid to the at least one array, the pre-filter being linked to a waste outlet of the device. The pre-filter may comprise filter gaps of about 20 μm. The device may comprise at least two sections of arrays of a plurality of isolation wells, the at least two sections being separated by a flow passage to a cell collection point receiving isolated cells from at least one of the sections of arrays. The device may comprise at least one section of arrays of a plurality of isolation wells. A pressure differential between the inlet and a waste outlet of the device may produce flow of the biological fluid through the at least one array of isolation wells to isolate the cells to be isolated. A reversed pressure differential between the inlet and a waste outlet of the device may produce a reversed flow of fluid in the device that permits retrieval of isolated cells. The cell trap may comprise an open flow side permitting a reversed flow of fluid to free an isolated cell from the trap. The cell trap may comprise a left or right tilted orientation, the at least one array of the plurality of isolation wells comprising a plurality of rows of isolation wells, and the plurality of rows of isolation wells comprising alternating left and right tilted orientations of the cell traps in successive rows of the plurality of rows of isolation wells. The cell trap may comprise at least three microstructures separated by at least two gaps between the at least three microstructures, the at least three microstructures forming a shape that includes a wider open side of the shape that is opposite a side of the shape in which the at least two gaps are situated.

In other related embodiments, the device may isolate the cells to be isolated from the biological fluid based solely on biorheological property differences between the cells to be isolated and the other components of the biological fluid. The device may permit retrieval of viable isolated circulating tumor cells from a blood sample. The cells to be isolated may comprise at least one of breast cancer cells, colorectal cancer cells, kidney cancer cells, lung cancer cells, gastric cancer cells, prostate cancer cells, ovarian cancer cells, squamous cell cancer cells, hepatocellular cancer cells and nasopharyngeal cancer cells. A pressure differential between the inlet and a waste outlet of the device may be within the range from about 5 kPa to about 15 kPa. Each of the at least one gaps may be about 4 μm to about 5 μm in width. The cell trap may be of a size and shape suitable to mechanically isolate a cell of between about 5 μm and about 40 μm in diameter. The cell trap may be crescent shaped and between about 15 lam and about 40 μm in its longer dimension, the longer dimension being a distance measured from the outermost edge of a tip of one horn of the crescent to the outermost edge of a tip of the other horn of the crescent, the outermost edges being the edges that are on an outer side of the crescent, which has a larger radius, as opposed to an inner bowl of the crescent, which has a smaller radius. The cell trap may comprise at least two gaps of about 4 μm to about 5 μm width. The cells to be isolated may comprise diseased cells. The cells to be isolated may comprise at least one of fetal cells, malaria infected cells, sickle anemia cells, dengue cells and stem cells. The cells to be isolated may comprise a diameter of between about 6 μm and about 25 μm. The device may be mounted on a microscope slide. The device on the microscope slide may be mounted on an inverted microscope. The device on the microscope slide may be mounted on an upright microscope. The device may permit an isolation efficiency of at least about 80% for the cells to be isolated. The cells to be isolated may comprise circulating tumor cells. The device may permit cell integrity of isolated cells to be preserved after isolation; and may permit retrieval of cancer cells at a prevalence on the order of about 1 cancer cell in about 1 ml of blood. No functional biochemical modification of the device or the cells to be isolated may be necessary to maintain integrity of isolated cells. The device may isolate cells based solely on physical properties of the cells to be isolated, the physical properties comprising at least one of shear modulus, stiffness, size and deformability. The cell trap may be of a size and shape suitable to mechanically isolate most often a single cell within the cell trap. The device may permit real time visualization of isolation of the cell. The device may further comprise an imaging system to capture images from the device to permit real time visualization of isolation of the cell. The device may permit real time enumeration of isolated cells. The device may further comprise an imaging system to capture images from the device to permit real time enumeration of isolated cells. The device may permit enumeration of isolated cells.

In another embodiment according to the invention, there is provided a method for isolating cells from a biological sample (e.g., a biological fluid). The method comprising flowing the biological fluid into an inlet of a microfluidic device; flowing the biological fluid from the inlet through at least one array of a plurality of isolation wells to isolate the cells to be isolated from the biological fluid, the cells being isolated by at least one isolation well of the plurality of isolation wells comprising a cell trap of a size and shape suitable to mechanically isolate a cell within the cell trap; and permitting components of the biological fluid, other than the cells to be isolated, to pass through the at least one array, the components being permitted to pass by at least one gap in the cell trap of a size and shape suitable to prevent passage of the cells to be isolated but to permit other components of the biological fluid to pass through the cell trap. The method may further comprise obtaining a biological sample from an individual.

In further, related embodiments, the biological fluid includes blood, lymph, spinal fluid, amniotic fluid, urine and the like. In a particular embodiment, the biological fluid may comprise blood, the cells to be isolated may comprise circulating tumor cells and the other components may comprise blood cells. The method may further comprise collecting isolated circulating tumor cells from the array of isolation wells at a cell collection point of the device; and passing waste blood cells from the isolation wells to a waste outlet of the device. The cell trap may comprise a crescent-shaped structure. The cell trap may comprise at least one of a “U” shaped structure, a “V” shaped structure and/or a “C” shaped structure. The method may comprise passing the biological fluid through a plurality of rows of isolation wells. The isolation wells of the plurality of rows of isolation wells may be offset from each other, for example by about 25 μm. The method may comprise passing the biological fluid through at least one row of isolation wells, the isolation wells of the at least one row being spaced apart from each other by a distance sufficient to prevent clogging of the at least one array by whole blood cells. The isolation wells of the at least one row may be spaced apart by about 50 μm.

In further, related embodiments, the method may comprise operating the inlet and a waste outlet of the device at a pressure differential that is within the range of physiological pressure differences found in circulating whole blood. The method may further comprise pre-filtering the biological fluid received from the inlet, flowing the pre-filtered biological fluid to the at least one array, and flowing waste from the pre-filtering to a waste outlet of the device. The pre-filtering may comprise flowing the biological fluid through filter gaps of about 20 μm. The method may comprise passing the biological fluid through at least two sections of arrays of a plurality of isolation wells, the at least two sections being separated by a flow passage to a cell collection point receiving isolated cells from at least one of the sections of arrays. The method may comprise passing the biological fluid through at least one section of arrays of a plurality of isolation wells. The method may comprise operating the inlet and a waste outlet of the device at a pressure differential that produces flow of the biological fluid through the at least one array of isolation wells to isolate the cells to be isolated; and may comprise reversing a pressure differential between the inlet and a waste outlet of the device to produce a reversed flow of fluid in the device that permits retrieval of isolated cells. The method may comprise reversing flow of fluid through the device to free an isolated cell from an open flow side of the cell trap.

In further, related embodiments, the cell trap may comprise a left or right tilted orientation, the at least one array of the plurality of isolation wells comprising a plurality of rows of isolation wells, and the plurality of rows of isolation wells comprising alternating left and right tilted orientations of the cell traps in successive rows of the plurality of rows of isolation wells. The cell trap may comprise at least three microstructures separated by at least two gaps between the at least three microstructures, the at least three microstructures forming a shape including a wider open side of the shape that is opposite a side of the shape in which the at least two gaps are situated. The method may comprise isolating the cells to be isolated from the biological fluid based solely on biorheological property differences between the cells to be isolated and the other components of the biological fluid. The method may comprise retrieving viable isolated circulating tumor cells from a blood sample. The method may comprise isolating viable circulating tumor cells comprising at least one of breast cancer cells, colorectal cancer cells, kidney cancer cells, lung cancer cells, gastric cancer cells, prostate cancer cells, ovarian cancer cells, squamous cell cancer cells, hepatocellular cancer cells and nasopharyngeal cancer cells.

In further, related embodiments, the method may comprise operating a pressure differential between the inlet and a waste outlet of the device within the range from about 5 kPa to about 15 kPa. Each of the at least one gaps may be about 4 μm to about 5 μm in width. The cell trap may be crescent shaped and between about 15 μm and about 40 μm in its longer dimension, the longer dimension being a distance measured from the outermost edge of a tip of one horn of the crescent to the outermost edge of a tip of the other horn of the crescent, the outermost edges being the edges that are on an outer side of the crescent, which has a larger radius, as opposed to an inner bowl of the crescent, which has a smaller radius. The cell trap may comprise at least two gaps of about 4 μm to about 5 μm width. The method may further comprise using the isolated cells to perform at least one of diagnosing and monitoring of a disease or condition of an individual in need of the at least one of diagnosis and monitoring. The isolated cells may comprise diseased cells. The isolated cells may comprise at least one of fetal cells, malaria infected cells, sickle anemia cells, dengue cells and stem cells. The cells to be isolated may comprise a diameter of between about 6 μm to about 25 μm. The method may further comprise performing an enumeration of the isolated cells. The method may comprise determining a cell type of the isolated cells. Determining the cell type may comprise staining the isolated cells within the microfluidic device. The stained isolated cells may be viewed under a microscope. The microfluidic device may be mounted on a microscope slide. The stained cells may comprise circulating tumor cells. The microfluidic device may be mounted on a microscope slide on an inverted microscope. The microfluidic device may be mounted on a microscope slide on an upright microscope. The method may comprise isolating the cells to be isolated with an isolation efficiency of at least about 80%. The cells to be isolated may comprise circulating tumor cells. The method may comprise preserving cell integrity of the cells to be isolated after isolation. The method may comprise retrieving cancer cells that have a prevalence on the order of about 1 cancer cell in about 1 ml of blood. The method may comprise performing no functional biochemical modification of the device or cells to be isolated while maintaining integrity of isolated cells. The method may comprise isolating cells based solely on physical properties of the cells to be isolated, the physical properties comprising at least one of shear modulus, stiffness, size and deformability. The cell trap may be of a size and shape suitable to mechanically isolate most often a single cell within the cell trap. The method may further comprise permitting real time visualization of isolating the cells. The method may further comprise capturing images from the device with an imaging system to permit real time visualization of isolating the cells. The method may comprise permitting real time enumeration of isolated cells. The method may further comprise capturing images from the device with an imaging system to permit real time enumeration of isolated cells.

In another embodiment according to the invention, there is provided a method of diagnosing cancer in an individual in need of diagnosis thereof. The method comprises flowing blood from a sample of the blood of the patient into an inlet of a microfluidic device; and flowing the blood from the inlet through at least one array of a plurality of isolation wells to isolate circulating tumor cells from the blood, wherein if circulating tumor cells are present the circulating tumor cells are isolated by at least one isolation well of the plurality of isolation wells comprising a cell trap of a size and shape suitable to mechanically isolate a circulating tumor cell within the cell trap. Blood cells are permitted to pass through the at least one array, the blood cells being permitted to pass by at least one gap in the cell trap of a size and shape suitable to permit blood cells to pass through the cell trap but to prevent passage of the circulating tumor cells to be isolated. The individual may be diagnosed with cancer based at least in part on the isolated circulating tumor cells.

In a further embodiment according to the invention, there is provided a method of monitoring progression of cancer in an individual in need of monitoring thereof. The method comprises flowing blood from a sample of the blood of the individual into an inlet of a microfluidic device; and flowing the blood from the inlet through at least one array of a plurality of isolation wells to isolate circulating tumor cells from the blood, the circulating tumor cells being isolated by at least one isolation well of the plurality of isolation wells comprising a cell trap of a size and shape suitable to mechanically isolate a circulating tumor cell within the cell trap. Blood cells are permitted to pass through the at least one array, the blood cells being permitted to pass by at least one gap in the cell trap of a size and shape suitable to permit blood cells to pass through the cell trap but to prevent passage of the circulating tumor cells to be isolated. Progression of the cancer in the individual may be monitored based at least in part on the isolated circulating tumor cells. The individual may be undergoing treatment for the cancer.

In a further embodiment, there is provided a method of monitoring treatment of cancer in an individual in need of monitoring thereof. The method comprises flowing blood from a sample of the blood of the individual into an inlet of a microfluidic device; and flowing the blood from the inlet through at least one array of a plurality of isolation wells to isolate circulating tumor cells from the blood, the circulating tumor cells being isolated by at least one isolation well of the plurality of isolation wells comprising a cell trap of a size and shape suitable to mechanically isolate a circulating tumor cell within the cell trap. Blood cells are permitted to pass through the at least one array, the blood cells being permitted to pass by at least one gap in the cell trap of a size and shape suitable to permit blood cells to pass through the cell trap but to prevent passage of the circulating tumor cells to be isolated. Treatment of the cancer in the individual may be monitored based at least in part on the isolated circulating tumor cells.

In a further, related embodiment, the method may further comprise determining the efficacy of the treatment based on the number of circulating tumor cells isolated by the microfluidic device.

In another embodiment according to the invention, there is provided a method of providing a prognosis of cancer in an individual in need of prognosis thereof. The method comprises flowing blood from a sample of the blood of the individual into an inlet of a microfluidic device; and flowing the blood from the inlet through at least one array of a plurality of isolation wells to isolate circulating tumor cells from the blood, the circulating tumor cells being isolated by at least one isolation well of the plurality of isolation wells comprising a cell trap of a size and shape suitable to mechanically isolate a circulating tumor cell within the cell trap. Blood cells are permitted to pass through the at least one array, the blood cells being permitted to pass by at least one gap in the cell trap of a size and shape suitable to permit blood cells to pass through the cell trap but to prevent passage of the circulating tumor cells to be isolated. Prognosis of the cancer in the individual may be provided based at least in part on the isolated circulating tumor cells.

In further, related embodiments, the method may further comprise retrieving the isolated circulating tumor cells from the micro fluidic device. The method may also further comprise identifying the isolated circulating tumor cells when they are situated within the microfluidic device.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is an overall view of a microdevice setup, in accordance with an embodiment of the invention.

FIG. 2 is a view of a microdevice setup showing fluid reservoirs connected to the microdevice, in accordance with an embodiment of the invention.

FIG. 3 is a view of fluidic connections to a microdevice in accordance with an embodiment of the invention.

FIG. 4 is an enlarged view of a microdevice in accordance with an embodiment of the invention.

FIG. 5 is a schematic drawing showing a design layout of isolation wells for circulating tumor cell trapping, in accordance with an embodiment of the invention.

FIG. 6 is a diagram of fabricated isolation well structures, in accordance with an embodiment of the invention.

FIG. 7 is an overall schematic diagram of a microdevice isolation chip, in accordance with an embodiment of the invention.

FIGS. 8A-8C are schematic diagrams of three variants of designs of isolation well layers, in accordance with an embodiment of the invention.

FIG. 9 is a diagram of a fabricated circulating tumor cell isolation device, in accordance with an embodiment of the invention.

FIG. 10 is a schematic diagram of a microdevice, showing the use of multiple sections of arrays of isolation wells separated by a flow passage, in accordance with an embodiment of the invention.

FIG. 11 is a close-up schematic diagram of isolation wells in a microdevice in accordance with an embodiment of the invention.

FIGS. 12A-12C are diagrams of a computational analysis of flow and shear stress around isolation wells under operating pressure, in accordance with an embodiment of the invention.

FIG. 13 is a graph showing the effects of input pressure on the estimated wall shear stress of an isolation cell in accordance with an embodiment of the invention.

FIG. 14 is a view of the cell isolation region of a microdevice, showing the successful arrest of MCF-7 breast cancer cells in the isolation structures, in accordance with an embodiment of the invention.

FIG. 15 is a graph of a quantitative analysis comparing cell isolation efficiency at various pressure settings for MCF-7, MDA-MB-231 and HT-29 cancer cells, in accordance with an embodiment of the invention.

FIG. 16 is a sequence of optical images depicting the retrieval of an arrested cell, in accordance with an embodiment of the invention.

FIG. 17 shows images of immunofluorescence staining results of an experiment investigating purity of isolated cancer cells in the microdevice, in accordance with an embodiment of the invention.

FIG. 18 is a graph of purity of cancer cell isolation over various operating pressures, in accordance with an embodiment of the invention.

FIGS. 19-21 are images showing a cell proliferation comparison between normal cultures (control) and retrieved cells in a microdevice in accordance with an embodiment of the invention.

FIG. 22 is a table showing the number of cancer cells isolated from low concentrations of spiked sample solutions at a pressure input of 5 kPa, in accordance with an embodiment of the invention.

### DETAILED DESCRIPTION OF THE INVENTION

A description of example embodiments of the invention follows.

Cancer metastasis is the main attribute to cancer-related deaths. Furthermore, clinical reports have shown a strong correlation between disease development and the number of circulating tumor cells (CTCs) in the peripheral blood of cancer patients.

In accordance with an embodiment of the present invention, there is provided a label-free microdevice capable of isolating cancer cells from whole blood via their distinctively different physical properties such as deformability and size. The isolation efficiency is on average at least 80% for tests performed on breast cancer, colon cancer and other types of cells. Viable isolated cells are also obtained which may give further insights to enhance the understanding of the metastatic process. Contrasting with conventional biochemical techniques, a microdevice according to an embodiment of the invention provides a mechanistic and efficient means of isolating viable cancer cells in blood. The microdevice has the potential to be used for routine monitoring of cancer development and cancer therapy in a clinical setting.

In accordance with an embodiment of the present invention, there is provided a device that presents a means to enumerate and isolate viable CTCs from peripheral blood with a high throughput and high efficiency. Isolation and quantification of CTCs presents an alternate disease marker that aid in monitoring cancer progression and determine overall survival. In accordance with an embodiment of the invention, retrieving viable CTCs assists in the further study of key biological determinants to the disease which might provide insights to the lethality of the disease.

Compared with most existing methodologies, the ability to separate CTCs from blood in fewer steps in accordance with an embodiment of the invention reduces the complexity of obtaining vital disease information. Most existing methodologies require multiple enrichment and identification procedures which is tedious and time-consuming. A device in accordance with an embodiment of the invention is able to take whole blood samples for processing directly.

Furthermore, viability of the cells is compromised in most existing approaches. Even in existing techniques using anti-EpCAM coated microstructures that are able to isolate viable CTCs, retrieval is a challenge because high shear flows are required to break the affinity of the biochemical bonds.

Microfluidic devices provide an alternative technique compared to conventional biochemical separations. Devices utilizing dielectrophoretic forces to separate and manipulate cells are advantageous as they do not require functionalization of the sample or the microdevice (P. Y. Chiou, A. T. Ohta, M. C. Wu, Nature 436, 370-372 (2005); D. S. Gray, J. L. Tan, J. Voldman, C. S. Chen, Biosens. Bioelectron. 19, 771-780 (2004); A. Rosenthal, J. Voldman, Biophys. J. 88, 2193-2205 (2005); J. Voldman, Curr Opin Biotechnol 17, (2006)). However, efficient cancer cell separation may be difficult due to the low concentration of CTCs in blood and the relative similar dimensions of leukocytes with cancer cells.

In accordance with an embodiment of the present invention, through a biomechanical means of isolation that utilizes CTCs' distinct biorheological difference with blood constituents, the retrieval of CTCs is made relatively easy to achieve by controlling the input/output conditions. In addition, no functional biochemical modification of the device or CTCs is necessary to maintain the integrity of CTCs. The microsystem has a high throughput and is able to process the blood samples within minutes. Further, an embodiment according to the invention permits real time visualization of the isolation process. Further, a system may provide a simple means of enumerating cells (such as CTCs) using a uniform array. In addition, a device may permit real time enumeration of isolated cells.

A device in accordance with an embodiment of the invention has potential application to aid clinicians in cancer disease monitoring and retrieval of viable CTCs for further analysis. The ease of use and portability of the device presents an attractive replacement to current methodologies. A high throughput ensures fast recovery time of diagnosis results which can aid in timely drug administering to better the chances of survival.

A device in accordance with an embodiment of the invention is fabricated in accordance with the biorheology of cells and depends on the deformability and dimensions of the cells. Studies of various different types of cells' rheology may be used to determine the dimensions of the device. In such studies, fresh cell samples may be used to ensure consistent rheological properties of the cells.

In accordance with an embodiment of the invention, there is provided a microfluidic device to isolate viable cancer cells of breast and colon origins from whole blood using solely the biorheological property differences of cancer cells and blood constituents. No functional modifications of the microdevice are required as isolation is solely dependent on the biorheological property differences of cancer cells and blood constituents. Past studies have revealed that the shear modulus, stiffness, size and/or deformability of diseased cells (L. Weiss, Adv Cancer Res 54, (1990); L. Weiss, D. S. Dimitrov, J. Theor. Biol. 121, 307 (1986)) is distinctively different from blood constituents (H. Mohamed, L. D. McCurdy, D. H. Szarowski, S. Duva, J. N. Turner, M. Caggana, IEEE Trans. Nanobioscience 3, 251 (2004); J. P. Shelby, J. White, K. Ganesan, P. K. Rathod, and D. T. Chiu, Proc. Natl. Acad. Sci. U.S.A. 100, 2003). The adopted approach draws upon this dissimilarity to achieve high purity in isolating cancer cells in blood. A feasibility study has also been successfully conducted to separate samples based on biorheological differences (S. J. Tan., L. Yobas, G. Y. H. Lee, C. N. Ong, C. T. Lim, International Conference on Biocomputation, Bioinformatics, and Biomedical Technologies, 2008. BIOTECHNO '08 (2008)). The isolation process is achieved in a single step, preserving the integrity and viability of these cells. Retrieval of the isolated cells is also straightforward by controlling and manipulating the flow conditions in a device according to an embodiment of the invention. The microdevice may be used to capture and isolate circulating tumor cells from peripheral blood of cancer patients for diagnostic and prognostic purposes. The microdevice is attractive for applications in oncology research, particularly prognostication and prediction of drug response.

An embodiment according to the invention overcomes the technical challenges posed by the low cancer cell count in blood combined with large sample volumes. By utilizing the biomechanical property differences of cancer cells from blood, an embodiment according to the invention achieves an effective isolation of cancer cells.

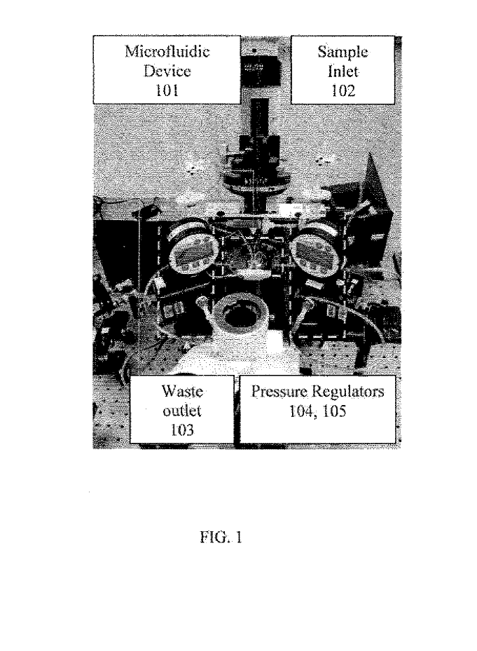

FIG. 1 is an overall view of a custom made experimental setup mounted on an inverted microscope, in accordance with an embodiment of the invention. A microfluidic device 101 has a sample inlet 102 from which the device receives a blood sample, a waste outlet 103 for receiving waste blood from which cancer cells have been isolated, and pressure regulators 104, 105 for regulating the inlet and outlet pressures of the device 101.

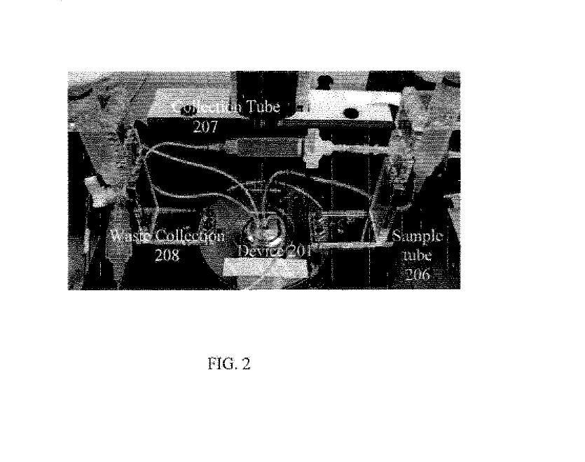

FIG. 2 is another view of a microdevice setup in accordance with an embodiment of the invention, showing fluid reservoirs connected to the device. The microdevice 201 receives blood from a sample tube 206, and isolates cancer cells for collection in collection tube 207. Waste is collected in waste collection 208.

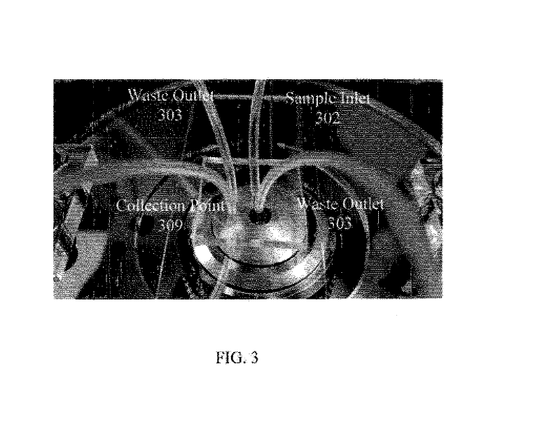

FIG. 3 is a view of fluidic connections to a microdevice in accordance with an embodiment of the invention. The microdevice is connected to a sample inlet 302, waste outlets 303 and fluid connections 309 to the collection point.

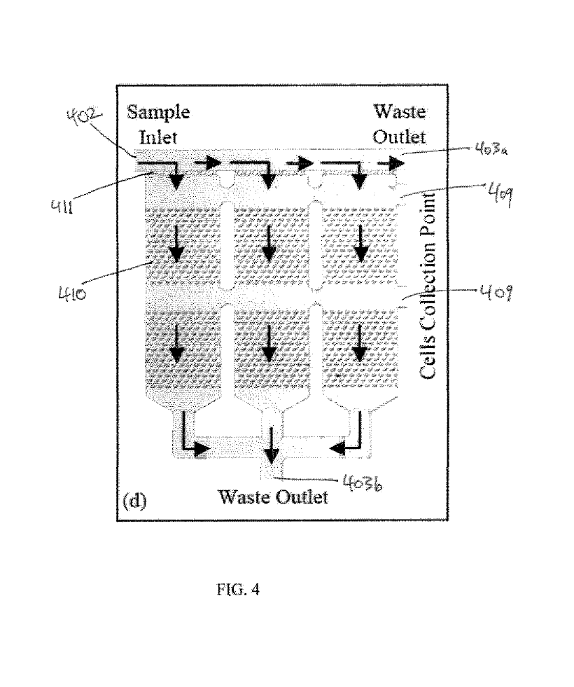

FIG. 4 is an enlarged view of a microdevice in accordance with an embodiment of the invention, showing a sample flow path during trapping of CTCs. The microdevice includes a plurality of arrays of cell isolation wells 410. The microdevice also includes pre-filters 411, which are used to prevent cell clumps from entering the device. The pre-filters may have gaps of width 20 μm, for example. The isolation wells 410 are arranged in rows, which may be offset from each other, for example by 25 μm, in order to enhance trapping efficiency. The arrows in FIG. 4 indicate the flow pattern through the microdevice during isolation of cells. The fluidic ports 402, 403a, 403b and 409 are pressure regulated which provides ease of control. Sample is injected into the sample inlet 402 at a designated pressure and is made to flow past the cell trapping structures (isolation wells 410). The distinctive biorheological difference in CTCs and blood constituents allow trapping of the CTCs in the traps 410 while blood constituents sieve through. Whole blood enters the sample inlet 402, and some flows directly to one of the waste outlets 403a. The rest is filtered through the pre-filters 411, and flows through the rows of isolation wells 410. Filtered blood is connected at the other of the waste outlets 403b. In the process of flowing the blood through the microdevice, cancer cells are isolated and trapped in the isolation wells 410. Through controlling the pressure differences at the various fluidic ports 402, 403a, 403b and 409, the sample flow in the device can be directed. Efficiency is affected in part by the pressure difference applied and the concentration of the cells in the sample. The arrays of physical traps (isolation wells 410) in the flow path impede the movement of CTCs while blood constituents sieve through the small gaps created in the structures of the isolation wells 410. To retrieve the trapped viable CTCs, the sample inlet 402 is blocked off and the cell collection point 409 is activated. Maintaining a pressure differential between the waste outlet 403b and the cell collection point 409, trapped cells flow into the cell collection tube 207 (FIG. 2) which can be reseeded for further analysis of the CTCs.

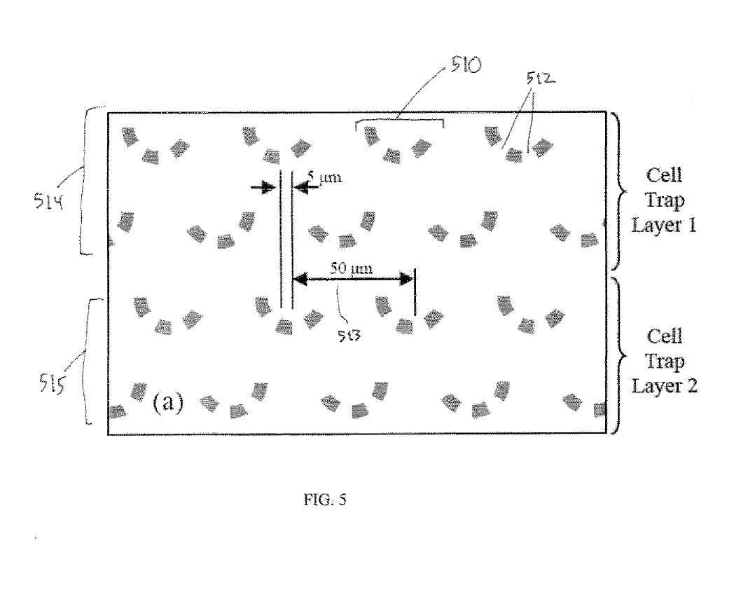

FIG. 5 is a schematic drawing showing the design layout of the isolation wells 510 for CTC trapping, in accordance with an embodiment of the invention. The isolation wells 510 have a crescent shape with gaps 512 that allow better trap efficiency by reducing fluidic resistance in that region. This effectively separates the CTCs from blood. The numerous crescent structures 510 may be positioned with a pitch 513 of, for example, 50 μm. Each layer of cell traps 514, 515 consists of two rows placed at an alternating position to each other. This permits a more efficient flow path to prevent clogging of the device. To increase the hydrodynamic efficiency of trapping CTCs, repeated patterns of trapping layers may be shifted, for example, 5 μm to the right with reference to the previous trap layer. For each crescent trap structure, two gaps 512 of, for example about 4 μm to about 5 μm may be present in order to better filter flood constituents. There may be, for example, a total of about 900 cell trap structures 510 per device. It will be appreciated that in accordance with an embodiment of the invention, other dimensions than provided herein may be used, depending for example on the dimensions and type of the cells to be isolated by the device.

In accordance with an embodiment of the invention, the sample inlet 402 (see FIG. 4) receives a biological fluid (such as blood) flowed into the device. One or more arrays of isolation wells 410 receive the biological fluid from the inlet 402. An isolation well includes a cell trap 510 (see FIG. 5) of a size and shape suitable to mechanically isolate a separate individual cell within the isolation well. The cell trap 510 includes one or more gaps 512 of a size and shape suitable to permit cells of the biological fluid to pass through the cell trap but to prevent passage of the cells to be isolated (such as circulating tumor cells). The cell trap 510 includes an open flow side (towards the top of the page in FIG. 5) permitting a reversed flow of fluid to free an isolated cell from the trap. Further, the cell traps 510 may have a left or right tilted orientation, and may alternate between left and right tilted orientations in successive rows (see FIG. 5; the top row has a right tilted orientation, the next row down has a left tilted orientation, and so forth).

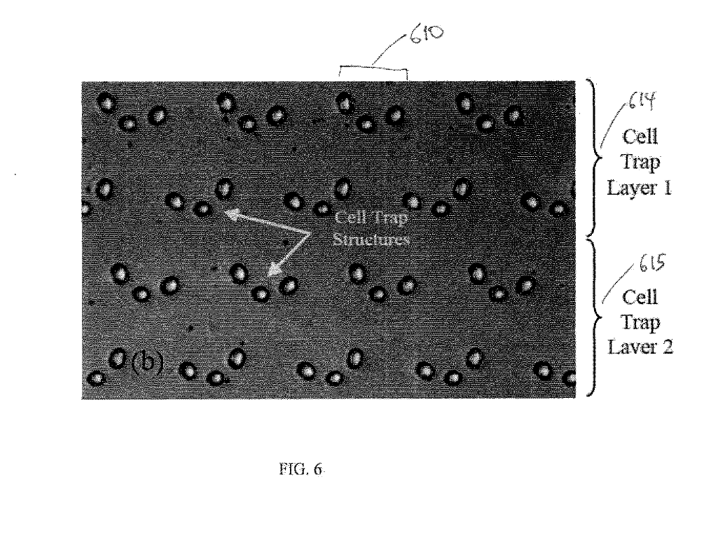

FIG. 6 is a diagram of fabricated isolation well structures 610 corresponding to the schematic of FIG. 5, in accordance with an embodiment of the invention. Two layers of isolation wells 614 and 615 are shown. The isolation well structures 610 (also called cell traps herein) have a crescent- or V-shaped structure using three microstructures separated by two gaps between the three microstructures. The three microstructures form a shape that includes a wider open side that is opposite the side in which the at least two gaps are situated. It will be appreciated that various possible structures may be used to achieve a similar effect. For example, a “crescent,” “C”, “U,” or “V” shaped structure may be used.

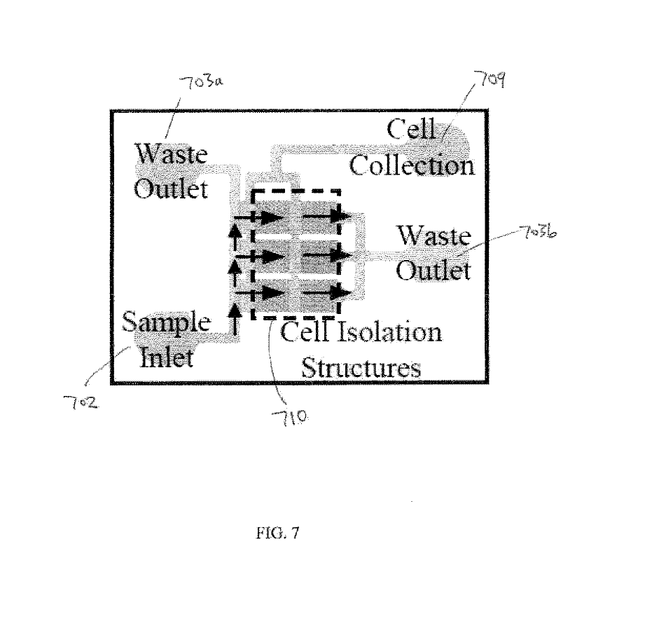

FIG. 7 is an overall schematic diagram of a microdevice isolation chip, in accordance with an embodiment of the invention. The microdevice includes a sample inlet 702, waste outlets 703a and 703b, cell collection point 709 and isolation wells 710. Arrows indicate the direction of flow through the isolation wells 710 during trapping of the circulating tumor cells. A pressure differential (for example, between the inlet 702 and waste outlet 703b) produces flow through the isolation wells 710 to isolate the circulating tumor cells. When it is desired to collect the isolated circulating tumor cells, a reversed pressure differential is applied to produce a reversed flow of fluid in the device, which permits retrieval of isolated cells at cell collection point 709.

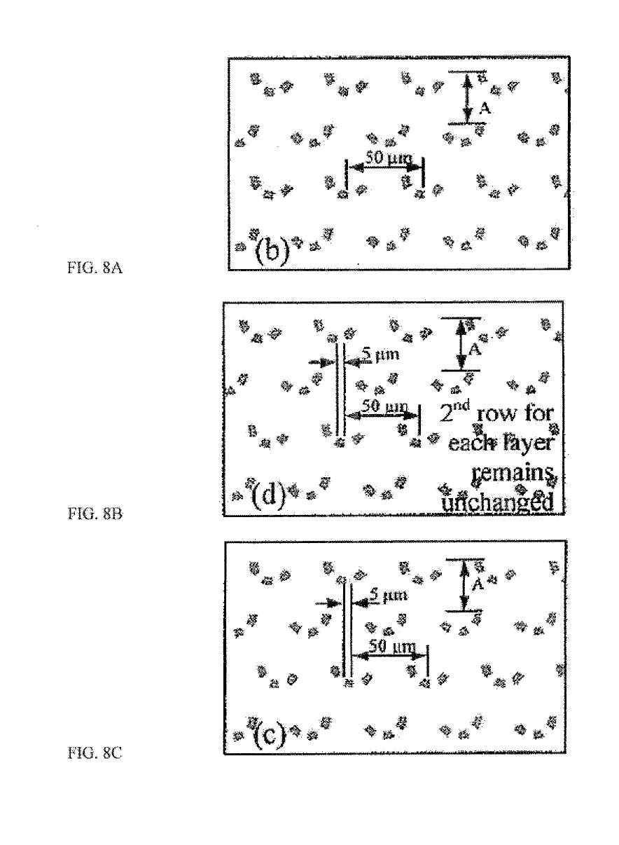

FIGS. 8A-8C are schematic diagrams of three variants of designs of isolation well layers, in accordance with an embodiment of the invention. Different variants may be used to achieve different efficiency in isolation of CTCs from peripheral blood. By changing the position of the cell trap layers, the three variants shown in FIGS. 8A-8C are obtained. FIG. 8A uses regular arrays of cell trap layers. FIG. 8B has the first row of each trapping layer shifted 5 μm to the right and while the second row remains unchanged. FIG. 8C has the entire cell trap layer shifted to the right by 5 μm. The shift of the traps theoretically enhances the hydrodynamic efficiency, thereby enabling better CTC capture rates. Another three variants could be produced by changing the dimension “A” between each row of cell traps. Other variations could be used. The initial design accounts for rigid samples, but dimensions can be reduced for deformable samples like other types of diseased cells and stem cells. Compressing the rows of cell traps has the advantage of making the fluidic resistance in the region more uniform, which enhances the probability of CTCs getting trapped.

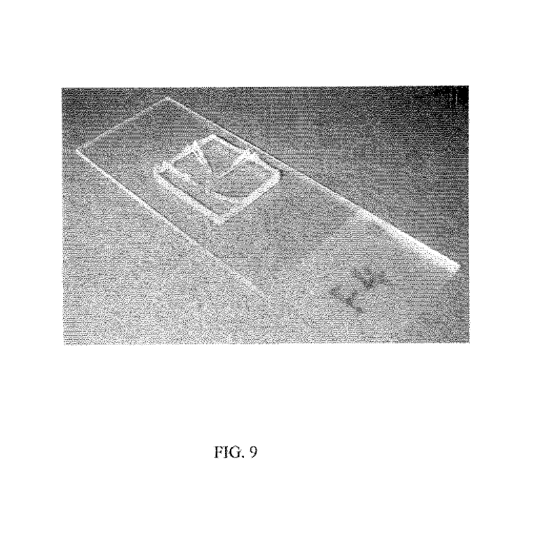

FIG. 9 is a diagram of a fabricated CTC isolation device, in accordance with an embodiment of the invention. The device may be fabricated in polydimethylsiloxane (PDMS) bonded onto glass. It will be appreciated that other materials may be used.

### Experimental: Microdevice Design and Fabrication

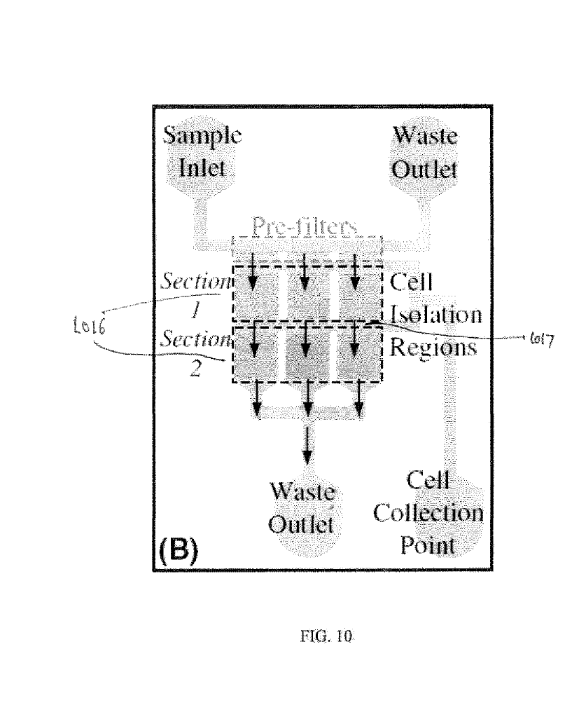

FIG. 10 is a schematic diagram of a microdevice, showing the use of multiple sections 1016 of arrays of isolation wells separated by a flow passage 1017, in accordance with an embodiment of the invention. The device may also use only one single section of arrays of isolation wells, or at least one section, or at least two sections.

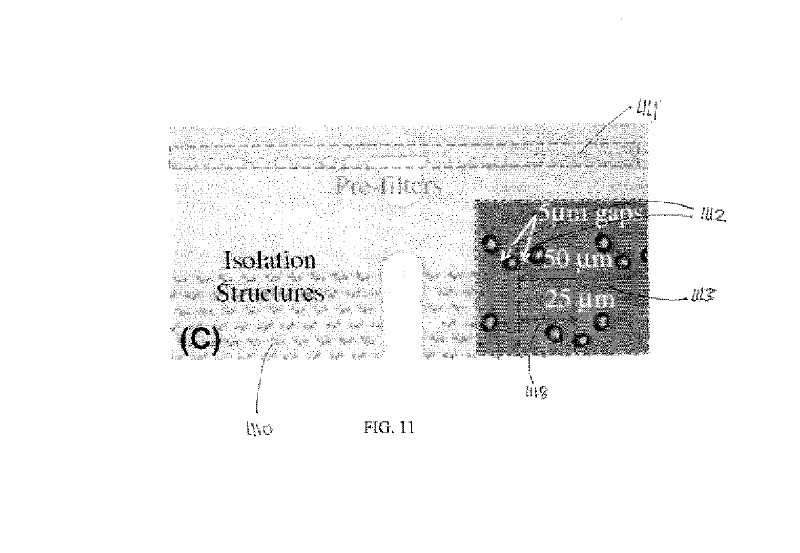

FIG. 11 is a close-up schematic diagram of isolation wells 1110 in a microdevice in accordance with an embodiment of the invention. The gaps 1112 of, for example, about 4 μm to about 5 μm in each of the traps ensure the exit of blood constituents due to the ability of blood constituents to traverse very small constrictions. In this way, larger white blood cells (WBCs) of comparable dimensions to, but more deformable than, cancer cells can be effectively removed and this will ensure a high purity of trapped cancer cells. Each trap is positioned with a pitch 1113 of, for example, 50 μm which effectively prevents cells from clogging in the microdevice. The pre-filters 1111 with gaps of 20 μm shown in FIG. 11 also serve to prevent larger clumps or debris from clogging up the setup and are linked to the waste outlet to effectively remove debris. Clogging prevention helps to obtain a feasible device and maximizing isolation purity helps to reduce false positive results, which is a likely encountered problem for devices that try to separate and isolate cells through physical means (D. Di Carlo, L. Y. Wu, L. P. Lee, Lab. Chip. 6, 1445 (2006); H. Mohamed, L. D. McCurdy, D. H. Szarowski, S. Duva, J. N. Turner, M. Caggana, IEEE Trans. Nanobioscience 3, 251 (2004); N. Pamme, Lab Chip 7, 1644 (2007)). In addition, each layer of isolation structures may be offset 1118 by 25 μm to enhance hydrodynamic efficiency. The isolation wells 1110 may be further divided into two sections (see 1016 of FIG. 10) that facilitated maximal retrieval of isolated cells. During cell retrieval, the flow direction is reversed from the indicated arrows shown in FIG. 10 and the rounded inverted crescent-shape of the isolation wells 1110 provide a favorable path to enable the cells to be extracted out to the cell collection point. The crescent-shaped isolation well structures also minimize physical interactions to reduce possible mechanical damage during retrieval. For each microdevice, there may be, for example, a total of 900 isolation well structures, although other numbers may be used.

In an experiment in accordance with an embodiment of the invention, the arrays of crescent-shaped structures were created using soft lithography (J. C. McDonald, G. M. Whitesides, Acc. Chem. Res 35, 491 (2002)). The photo mask was drawn in Cadence (Cadence Design Systems, Inc., San Jose, Calif., USA) and produced on glass with critical dimensions of 2 μm (Infinite Graphics Inc., Minneapolis, Minn., USA). Fabrication of the masters was done by spin coating (3200 rpm, 45 seconds) SU-8 2025 (MicroChem Corp., Newton, Mass., USA) on an 8-inch silicon wafer. Depth of the microdevice was 18 μm and it was cast from polydimethylsiloxane (PDMS) (Sylgard 184, Dow Corning Corp., Midland, Mich., USA). Following manufacturer's instructions, pre-cured PDMS was degassed in a desiccator and cured in an oven preset at 80° C. for 2 hours. After peeling the PDMS off the master, fluidic ports were punched using a flat tip needle. Irreversible bonding of the PDMS microdevice to a glass slide was achieved using oxygen plasma treatment.

It will be appreciated that other techniques of fabrication may be used in accordance with an embodiment of the invention.

### Experimental: Computational Fluid Dynamics (CFD) Analysis

In accordance with an embodiment of the invention, a 3D computational model of the microdevice was developed to better understand the flow characteristics as well as to help in the design of the microdevice. A simplified model of the microdevice consisting of fourteen isolation structures was created using Gambit (Ansys Inc., Lebanon, N.H., USA) and simulated using Fluent (Ansys Inc., Lebanon, N.H., USA). An optimized mesh number of 579,820 was used. Mesh independence was ascertained by increasing the mesh number and observing the difference in velocities to be less than 1%. Adopting a no-slip wall boundary condition and fluid properties of pure H₂O (density at 998.2 kg/m³ and viscosity 0.001003 kg/ms), the study was carried out by analyzing the velocity profiles and shear stresses at the irregular isolation structures under different initial conditions.

With the computational results, simulated particles could be traced in the flow to check upon the isolation effectiveness of the microdevice. 25 simulated particles were placed uniformly at the inlet and the flow pathlines traced to check whether the particles intercept the crescent traps. Where the pathlines intercepted with the crescent trap, it was presumed that there was a possibility for cells in the sample solution to be impeded. This helped to determine if the design was able to fulfill its task effectively. When simulating in the reverse flow direction for cell retrieval, the flow pathlines of the simulated particles aided to determine whether the isolation structures obstruct the recovery. This helps to ensure minimal damage to the isolated cells. The shear stresses and flow patterns in the microdevice at various operating conditions were extracted from the computational analysis and compared to physiological conditions to aid in optimizing the design and determining the operating conditions for the microdevice.

### Experimental: Cell Culture and Cell Size Measurements

In accordance with an embodiment of the invention, three different cell lines MCF-7, MDA-MB-231 (human breast adenocarcinoma) and HT-29 (human colon adenocarcinoma) of human origin were tested in the microdevice. Culturing of cells was done in a 25 cm² tissue culture flask and maintained with Dulbecco's Modified Eagle Medium (DMEM) (Sigma, St. Louis, Mo., USA) supplement with 10% fetal bovine serum (FBS) (Hyclone, Logan, Utah, USA) and 1% penicillin G/streptomycin/amphotericin B (Gibco, Carlsbad, Calif., USA). For each experiment, cells were grown to confluence, trypsinized and resuspended in culture media. A portion of the suspended cells were taken as control in the experiment and cultured normally. The rest were diluted to a concentration of approximately 100 cells per milliliter and the sample solution was injected into the device for characterizing its isolation efficiency. In the cell proliferation analysis, isolated cells were retrieved from the microdevice and reseeded into the culture flask to observe their proliferation status over a period of 5 days under normal culturing conditions.

For cell size measurements of the cancer cell lines, the diameter was an average reading obtained from images of 100 suspended cells and determined using an image processing software (NIS-Elements AR, Nikon Corp., Singapore). Cancer cell counting was done with a hemocytometer and serial diluted to achieve the desired concentration of 100 cells per milliliter of 1× phosphate buffered saline (PBS).

### Experimental: Sample and Apparatus Preparation

In an experiment in accordance with an embodiment of the invention, all apparatus including the microdevices, tubes and fluidic connectors, were thoroughly sterilized with 70% ethanol prior to use. Subsequently, sterile 1×PBS was injected to wash out any traces of ethanol. The microdevice was then mounted in a custom made fixture with the fluidic connections attached (see the embodiment of FIG. 1). The fixture allowed portability of the setup and positioned the sample solution close to the microdevice to minimize sample wastage. To control the flow in the microdevice, two pressure regulators 104, 105 were used to create the pressure differential between the inlet 102 and the waste reservoir 103. The use of pressure regulation enables instantaneous and real time changes to the flow characteristics in the device.

To ascertain the isolation purity, each of the cancer cell types were added into whole blood donated from healthy donors at concentration of approximately 100 cells per milliliter. The sample solution was further diluted with sterile 1×PBS in a 1:2 ratio to reduce the sample viscosity so that it could be processed easily. Isolated cells in the microdevice were stained with fluorescence (see below) to distinguish between cancer cells and WBCs. For cell proliferation experiments, isolated cells were retrieved by reversing the pressure differential and directing the isolated cells to the collection point. The collected solution was then centrifuged at 1200 rpm for 5 minutes with the cell pellet resuspended later in culturing media DMEM and reseeded in a T25 culture flask. Their proliferative rates were compared to normal cultures which acted as controls in the experiment. A further part of the analysis involved testing the microdevice at lower concentrations of cancer cells in sterile 1×PBS (1-2 cells per milliliter). Cells were picked out manually using a pipette with a 200 μl pipette tip from a cell suspension of approximately 100 cells per milliliter under the microscope. The cells were then directly added to 1×PBS and injected into the microdevice.

### Experimental: Immunofluorescence Staining to Identify CTCs

In an experiment in accordance with an embodiment of the invention, immunofluorescence staining allowed visual examination to characterize cancer cell isolation purity in the microdevice. For the control experiment, the premixed sample of blood and cancer cells (200 μl) was incubated onto a 12 mm coverslip (polylysine coated) for 30 minutes. The sample was subsequently fixed with 4% paraformaldehyde (PFA) for 30 min and washed with 1×PBS for 15 minutes. It was then permeabilized by 0.1% Triton X-100 in 1×PBS for 10 minutes, followed by a PBS wash for 15 minutes and adding 10% goat serum for 30 minutes to block out non-specific bindings. The sample was then stained for Epithelial Cell Adhesion Molecule (EpCAM) (1:50, Santa Cruz Biotechnology Inc., Santa Cruz, Calif., USA) for 1 hour followed by the secondary antibody (1:500, goat anti-mouse AlexaFluor 568, Invitrogen Corp., Carlsbad, Calif., USA) for another hour to identify cancer cells. Fluorescein isothiocyanate (FITC) conjugated CD45 (1:50, Santa Cruz Biotechnology Inc., Santa Cruz, Calif., USA) was then used (1 hour) to identify WBCs and 4′,6-diamidino-2-phenylindole (DAPI) for nuclei visualization. The coverslip was then washed with 1×PBS for 10 minutes and mounted.

For staining in the microdevice, a pressure differential of 5 kPa was used to induce flow into the microdevice. The value of 5 kPa was chosen as it best preserves the state of the isolated cells in the microdevice as compared to using higher pressure differentials. Lower pressure conditions will increase the processing time. Captured cells were first fixed by flowing 4% PFA for 30 minutes, permeabilized by 0.1% Triton X-100 in 1×PBS for 10 minutes, followed by washing with 1×PBS for 15 minutes and adding 10% goat serum to block out non-specific bindings for 30 minutes. To identify cancer cells, 0.2 ml of EpCAM antibodies were injected for 15 minutes, left to stand for another 45 minutes and followed by PBS washing. The procedures of antibody injection and PBS wash were repeated for the secondary antibody (1:500, goat anti-mouse AlexaFluor 568, Invitrogen Corp., Carlsbad, Calif., USA). For the identification of WBCs, 0.2 ml of FITC conjugated CD45 antibodies were injected for 15 minutes, left to stand for another 45 minutes and followed by washing with PBS. Staining was completed by flowing DAPI for 15 minutes at 5 kPa followed by washing with PBS.

### Experimental: Clinical Significance and CFD Analysis

The prognostic values of enumerating CTCs in peripheral blood have been widely reported (P. D. Beitsch, E. Clifford, Am. J. Surg. 180, 446 (2000); G. T. Budd, M. Cristofanilli, M. J. Ellis, A. Stopeck, E. Borden, M. C. Miller, J. Matera, M. Repollet, G. V. Doyle, L. W. Terstappen, D. F. Hayes, Clin. Cancer Res. 12, 6403 (2006); M. Cristofanilli, K. R. Broglio, V. Guarneri, S. Jackson, H. A. Fritsche, R. Islam, S. Dawood, J. M. Reuben, S. W. Kau, J. M. Lara, S. Krishnamurthy, N. T. Ueno, G. N. Hortobagyi, V. Valero, Clin. Breast Cancer 7, 471 (2007); H. Gogas, G. Kefala, D. Bafaloukos, K. Frangia, A. Polyzos, D. Pectasides, D. Tsoutsos, P. Pangiotou, J. Ioannovich, D. Loukopoulos, Br. J. Cancer 87, 181 (2002); D. F. Hayes, M. Cristofanilli, G. T. Budd, M. J. Ellis, A. Stopeck, M. C. Miller, J. Matera, W. J. Allard, G. V. Doyle, L. W. Terstappen, Clin. Cancer Res. 12, 4218 (2006); D. S. Hoon, Nat. Clin. Pract. Oncol. 1, 74 (2004)). This method is also minimally invasive as compared to traditional biopsies. The number of CTCs in blood is directly associated with the disease progression and can help in evaluating cancer treatment efficacy (F. Nole, E. Munzone, L. Zorzino, I. Minchella, M. Salvatici, E. Botteri, M. Medici, E. Verri, L. Adamoli, N. Rotmensz, A. Goldhirsch, M. T. Sandri, Ann. Oncol. 19, 891 (2008)). Thus, it is important to have a high cancer cell isolation efficiency for the microdevice to count these rare cells in blood samples accurately. An approach in accordance with an embodiment of the invention draws mainly upon the highly deformable nature of erythrocytes (J. P. Shelby, J. White, K. Ganesan, P. K. Rathod, and D. T. Chiu, Proc. Natl. Acad. Sci. U.S.A. 100, 2003) and leukocytes (B. Yap, and R. D. Kamm, J. Appl. Physiol. 98, (2005)) that enable these cells to traverse capillaries as small as 2-5 μm whose cell diameters can range from 8 to 25 μm. On the other hand, cancer cells are more likely to be arrested in capillaries of similar dimensions (L. Weiss, D. S. Dimitrov, Cell Biophys. 6, 9 (1984)). Average sizes of MCF-7, MDA-MB-231 and HT-29 cells used in the experiments in accordance with an embodiment of the invention were tabulated to be 16.2±1.80 μm, 17.9±2.94 μm and 15.5±1.25 μm respectively. It should be noted that these size variances were for cell lines; actual clinical samples typically have higher variation in size. For example, renal cancer cells may have a size of from about 5 μm or about 6 μm to about 15 μm.

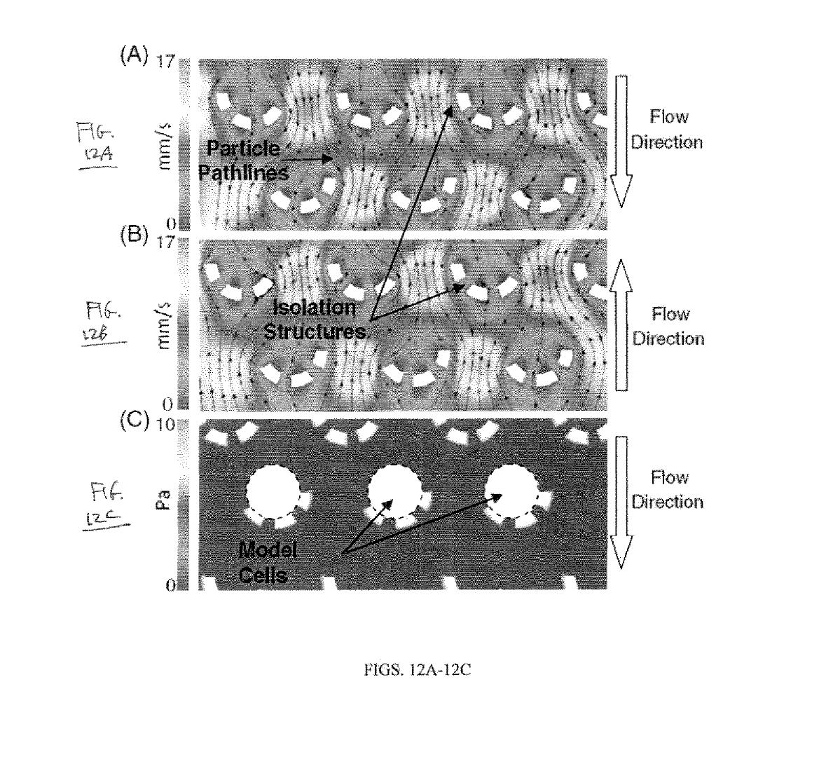

In an experiment in accordance with an embodiment of the invention, in order to understand the flow profile around the irregular shaped structures and ascertain minimal damage on isolated cancer cells due to hydrodynamic forces, the fluid velocity and shear stress profiles were simulated for the operating pressure differentials applied. FIGS. 12A-12C are diagrams of a computational analysis of flow and shear stress around isolation wells at an operating pressure of 5 kPa, in accordance with an embodiment of the invention.

FIG. 12A is a velocity profile when isolating cells taken at mid-plane (10 μm from the base) of the model, in accordance with an embodiment of the invention. In isolating cancer cells, the gaps facilitated the entry of the cells as shown by the traced path lines of the simulated particles in FIG. 12A. Flow velocities were also much lower near the isolation regions. In addition, the flow path was diverted when the trap was occupied which prevented clogging. The crescent shape of the isolation well which is alternated left and right aided in preventing clogging and allowed each isolation well to hold a single cell. Due to the heterogeneous nature of the cell sample, the cell sizes had a relatively significant variation. Traps occupied by smaller cells tended to be able to hold more than one cell. With this design, it helps to divert incoming cells to the next level to prevent obstruction in that region. This was verified with experimental observations showing the ability of the microdevice to effectively direct the cells away from occupied traps.

FIG. 12B is a velocity profile when retrieving cells, in accordance with an embodiment of the invention. For the purposes of cell retrieval, the flow direction was reversed by applying a positive pressure differential across the waste outlet and cell collection point. During cell retrieval, the inverted isolation structures enabled a streamline profile that minimizes impediment to the cells, thereby ensuring a high percentile of retrieval. The rounded base of the crescent traps also helped to minimize obstructions to the cells during retrieval to prevent external trauma which may be detrimental to the isolated cells (S. F. Chang, C. A. Chang, D. Y. Lee, P. L. Lee, Y. M. Yeh, C. R. Yeh, C. K. Cheng, S. Chien, J. J. Chiu, Proc. Natl. Acad. Sci. USA 105, 3927 (2008)).

FIG. 12C is a diagram showing shear stress acting on a spherical cell model when the cells are arrested in the isolation structures, in accordance with an embodiment of the invention. Contrasting to the physiological states experienced in large arteries (1.0-7.0 Pa) (A. M. Malek, S. L. Alper, S. Izumo, JAMA 282, 2035 (1999)), the estimated average wall shear stress around the isolated cells due to the flow was comparable (1.80 Pa). This indicates that the cells are likely to maintain their integrity while in the microdevice.

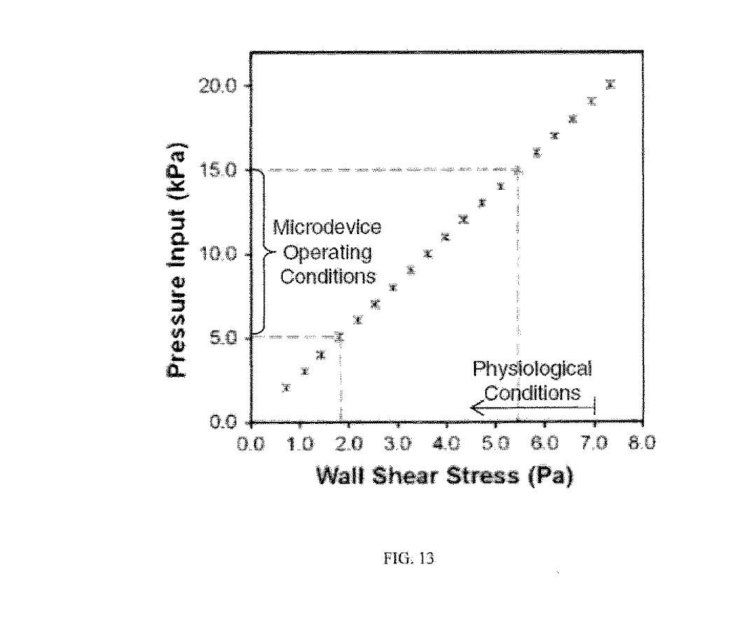

FIG. 13 is a graph, obtained using simulation studies, showing the effects of input pressure on the estimated wall shear stress of an isolation cell in accordance with an embodiment of the invention. The operating range of 5−15 kPa was selected for driving the flow in the microdevice so that the wall shear stress was within the range of physiological conditions.

### Experimental: Cancer Cell Isolation Efficiency

In an experiment in accordance with an embodiment of the invention, for characterizing cell isolation efficiency, low concentration of cancer cells (100 cells per milliliter) spiked in 1×PBS was injected into the microdevice at various pressure differentials. Small numbers of cancer cells in the sample solution mimicked the rarity of CTCs in peripheral blood. By visually counting the number of trapped and escaped cancer cells, the efficiency of cancer cell capture could be determined using Equation (1).

Isolation Efficiency(%)=((Trapped Cells)/(Trapped Cells+Escaped Cells))×100% (1)

In an embodiment according to the invention, the main factor affecting cell isolation efficiency was the pressure applied, since the input condition directly alters the flow conditions in the device. Although higher flow rates mean less sample processing time, the larger shear forces on the cells are undesirable as they can cause cell behavior modifications due to mechanical activated signal pathways or cell death (S. F. Chang, C. A. Chang, D. Y. Lee, P. L. Lee, Y. M. Yeh, C. R. Yeh, C. K. Cheng, S. Chien, J. J. Chiu, Proc. Natl. Acad. Sci. USA 105, 3927 (2008)). In the experimental investigation, the selected pressure differentials included 5 kPa, 10 kPa and 15 kPa which are comparable to physiological conditions. However, it should be appreciated that other pressure differentials may be used depending on the types of cells to be isolated, which may be cancer cells or non-cancer cells.

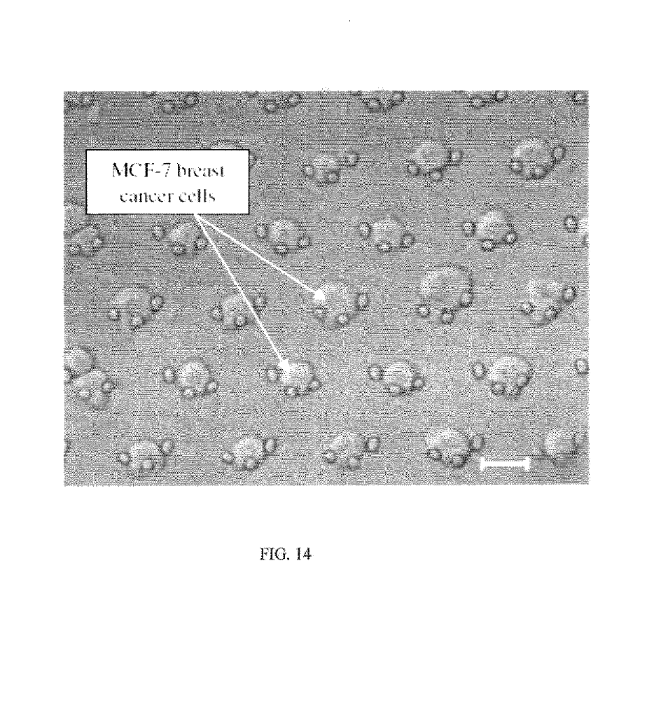

FIG. 14 is a view of the cell isolation region of a microdevice, showing the successful arrest of MCF-7 breast cancer cells in the isolation structures, in accordance with an embodiment of the invention. Mostly single cells were trapped in each crescent-shaped structure; the scale bar represents 20 μm. The single or doublet cells arrested in each isolation well also facilitated cell counting with ease. In accordance with an embodiment of the invention, a cell trap may be of a size and shape suitable to mechanically isolate most often a single cell within the cell trap, although it will be appreciated that occasionally more than one cell (such as two cells) are isolated in the cell trap. Using two pressure regulators connected to the sample inlet and the waste outlet, the pressure differential could be kept stable and used to maintain constant flow condition.

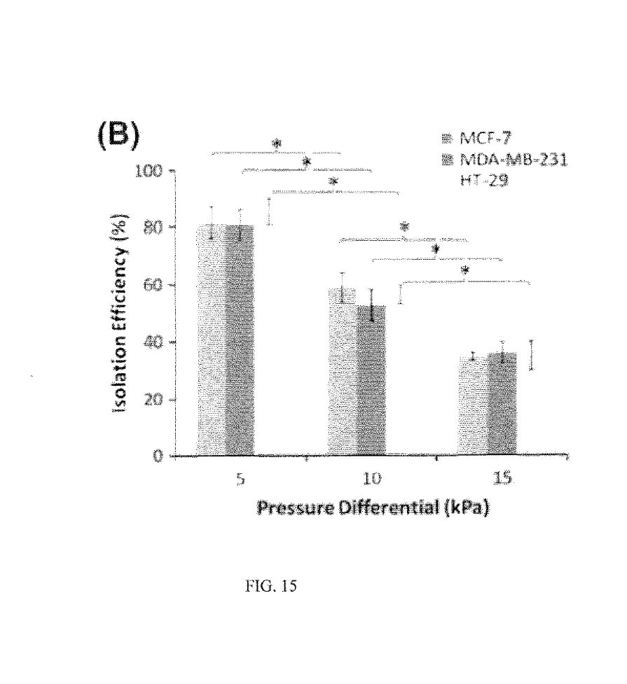

FIG. 15 is a graph of a quantitative analysis comparing cell isolation efficiency at various pressure settings for MCF-7, MDA-MB-231 and HT-29 cancer cells, in accordance with an embodiment of the invention. The graph shows microdevice isolation efficiency as a function of the input conditions. A total of 70 experimental runs were tabulated and error bars indicate measurement standard deviation; the “*” symbol refers to p<0.01 with a Student's t-test. The results favor the lower input pressure of 5 kPa to effectively isolate cancer cells with at least 80% isolation efficiency for all tested samples. The Student's t-test verifies that isolation efficiencies at 5 kPa are significantly higher (p<0.01) for all three samples than at higher pressure inputs. The reduction in efficiency as pressure differential increases can be accounted for by the dislodgement of the arrested cells due to increased hydrodynamic forces acting on these cells at higher pressure differentials. Experimental observations also indicate that smaller cancer cells are likely to be displaced after being momentarily trapped at higher pressure settings. At the pressure setting of 5 kPa, the microdevice was capable of processing samples at 0.71 ml/hr with a high isolation efficiency needed for an accurate diagnosis.

Other leading techniques to enrich cancer cells from peripheral blood have efficiency ranging from 20% to 90% (W. J. Allard, J. Matera, M. C. Miller, M. Repollet, M. C. Connelly, C. Rao, A. G. Tibbe, J. W. Uhr, L. W. Terstappen, Clin. Cancer Res. 10, 6897 (2004); M. Balic, N. Dandachi, G. Hofmann, H. Samonigg, H. Loibner, A. Obwaller, A. van der Kooi, A. G. Tibbe, G. V. Doyle, L. W. Terstappen, T. Bauernhofer, Cytometry B Clin. Cytom. 68, 25 (2005); O. Lara, X. Tong, M. Zborowski, J. J. Chalmers, Exp. Hematol. 32, 891 (2004)). However, there are also numerous restrictions and complex preparation procedures. For example, there is limited purity when detecting low concentrations of CTCs (D. A. Smirnov, D. R. Zweitzig, B. W. Foulk, M. C. Miller, G. V. Doyle, K. J. Pienta, N. J. Meropol, L. M. Weiner, S. J. Cohen, J. G. Moreno, M. C. Connelly, L. W. Terstappen, S. M. O'Hara, Cancer Res. 65, 4993 (2005)) in peripheral blood and various preparatory steps such as centrifugation, incubation and functional modifications which can be tedious and time consuming. A microdevice in accordance with an embodiment of the present invention is comparable to other leading biochemical techniques in terms of cancer cell enumeration from blood and is done without any functional modifications. Removing isolated cells for further analysis can also be achieved with ease by altering the flow conditions to induce the cells to flow towards the cell collection point.

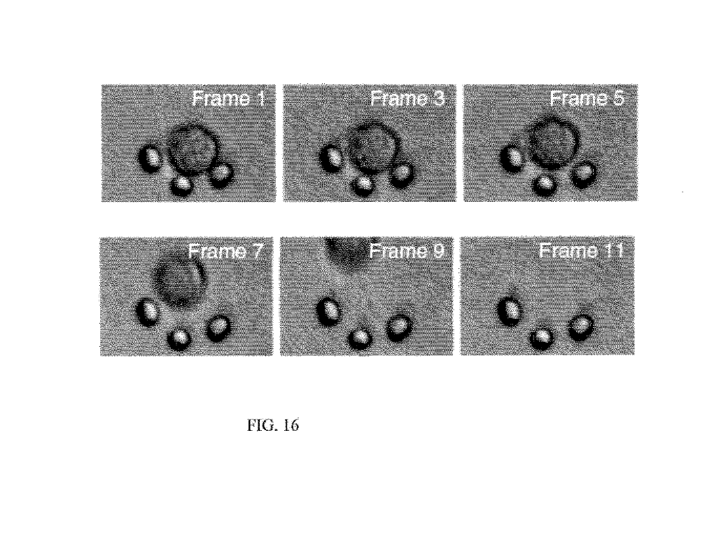

FIG. 16 is a sequence of optical images depicting the typical retrieval of one of the arrested cells, in accordance with an embodiment of the invention. The images of FIG. 16 are time sequence image of retrieving an isolated MDA-MB-231 cell, taken at approximately 110 fps (period of 0.1 sec).

### Experimental: Cancer Cell Isolation Purity

In an experiment in accordance with an embodiment of the invention, in order to ascertain the capture purity, cancer cells mixed with blood samples collected from healthy human volunteers (−100 cancer cells per milliliter) were diluted with 1×PBS to reduce sample viscosity and injected into the microdevice. To differentiate between hematologic and cancer cells, immuno-fluorescence staining of the isolated cells was carried out. It is reported that the EpCAM is over-expressed in human carcinoma (P. A. Baeuerle, O. Gires, Br. J. Cancer 96, 417 (2007); W. A. Osta, Y. Chen, K. Mikhitarian, M. Mitas, M. Salem, Y. A. Hannun, D. J. Cole, W. E. Gillanders, Cancer Res. 64, 5818 (2004)) which makes this an ideal marker to identify the cancer cells. MCF-7, MDA-MB-231 and HT-29 have been reported to be positive for EpCAM (D. Flieger, A. S. Hoff, T. Sauerbruch, I. G. Schmidt-Wolf, Clin. Exp. Immunol. 123, 9-14 (2001); W. A. Osta, Y. Chen, K. Mikhitarian, M. Mitas, M. Salem, Y. A. Hannun, D. J. Cole, W. E. Gillanders, Cancer Res. 64, 5818 (2004)). CD45, a trans-membrane glycoprotein is expressed among hematologic cells and was used to distinguish WBCs.

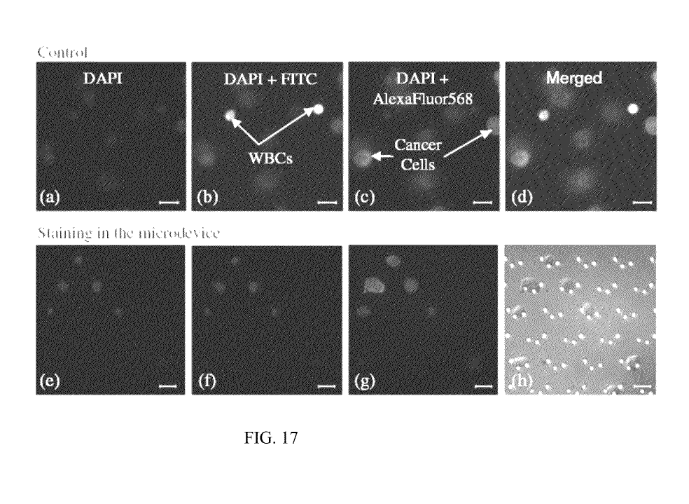

FIG. 17 shows images of immunofluorescence staining results of an experiment investigating purity of isolated cancer cells in the microdevice, in accordance with an embodiment of the invention. Immunofluorescence staining was performed on the control experiment (a-d in FIG. 17) and isolated cells in the microdevice (e-h in FIG. 17) to identify cancer cells and WBCs. EpCAM was stained with red fluorescence (cancer cells), CD45 with green fluorescence (WBCs) and nuclei materials with blue fluorescence. The scale bar represents 20 μm. FIG. 17 depicts that high purity can be achieved, showing the absence of WBCs, with no visible WBC fluorescence in the isolated cells in the microdevice (e-h in FIG. 17).

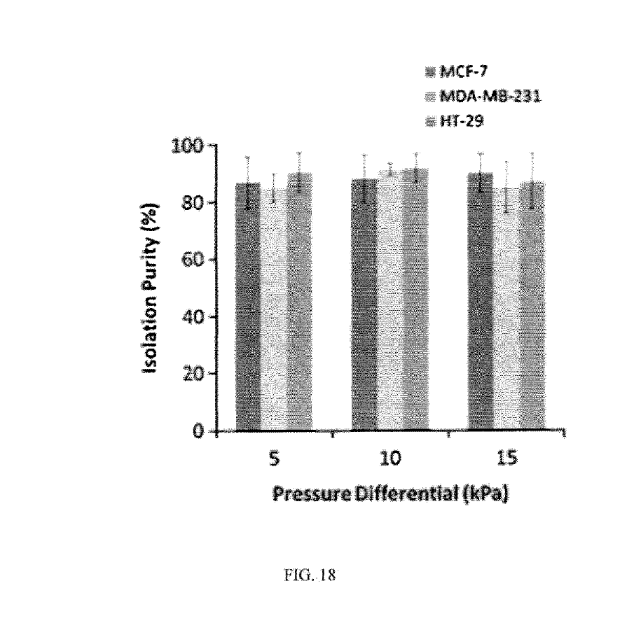

FIG. 18 is a graph of purity of cancer cell isolation over various operating pressures, in accordance with an embodiment of the invention. It can be seen that the purity is maintained over the entire pressure range, showing no significant difference in a Student's t-test for MCF-7, MDA-MB-231 and HT-29 cancer cells at p<0.01. The purity was calculated by the ratio of cancer cells to the total number of cells isolated from the blood mixture. This is also significantly higher than leading techniques which claim a separation purity of approximately 50% using biochemical means (S. Nagrath, L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, M. Toner, Nature 450, 1235 (2007)).

### Experimental: Conditions of Isolated Cells and Cell Retrieval

In an experiment in accordance with an embodiment of the invention, the conditions of cancer cells after isolation were of interest given that tumor cells in circulation are likely to be responsible for the progression of the disease cancer. Preserving the native state of cells after isolation will help to determine their exact nature and allow a detailed study of CTC sub-populations. Retrieval of the isolated cells in the microdevice was achieved by altering the inlet conditions with the pressure regulators. The waste reservoir was cleared first to prevent backflow of waste materials, followed by closing of the sample inlet fluidic port. The valve leading to the cell collection point was then opened and the pressure differential between the waste outlet and cell collection point quickly increased to 20 kPa. Isolated cells were then dislodged and the recovery rate and standard deviation were determined to be (95±8.0) % for MCF-7, (97±2.6) % for MDA-MB-231 and (96±4.4) % for HT-29. The recovery rate was calculated based on the number of cells that were dislodged to the initial number of trapped isolated cells. The process of cell recovery was repeated for 5-8 cycles to obtain enough cell number and the retrieved cells were then reseeded to a culture flask.

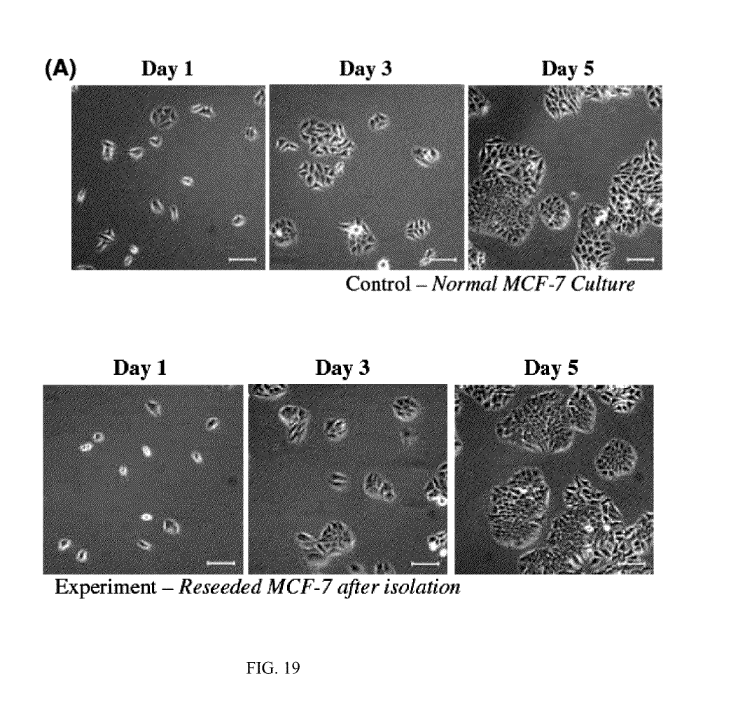

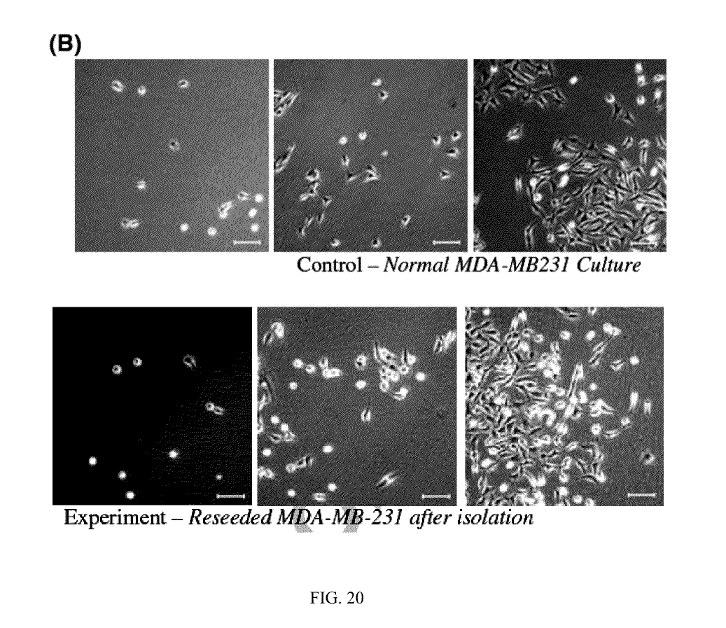

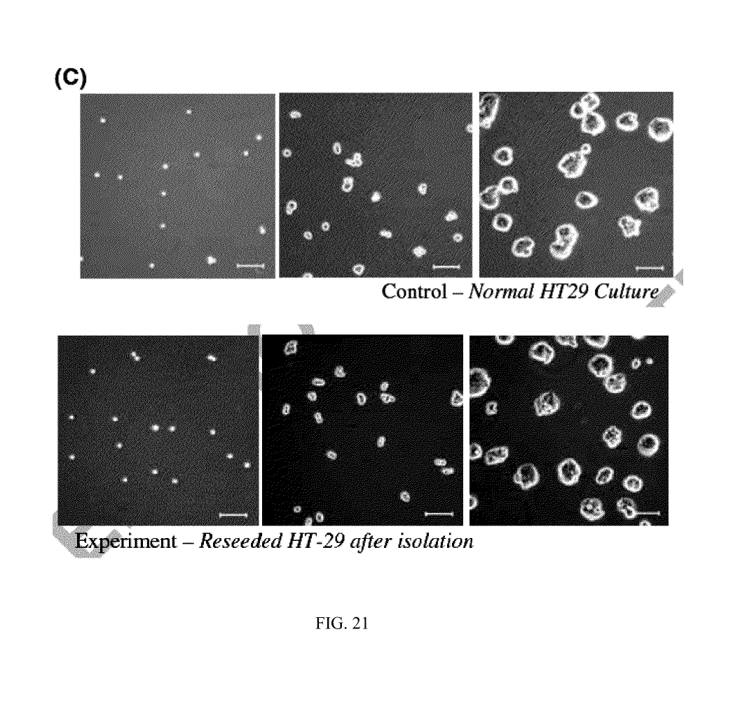

FIGS. 19-21 are images showing a cell proliferation comparison between normal cultures (control) and retrieved cells in a microdevice in accordance with an embodiment of the invention. The proliferative rates of reseeded cells were compared to normal cultured cells which acted as controls to ascertain whether isolated cancer cells are not affected by the microdevice. FIGS. 19-21 illustrate an overview of a 5-day culture for MCF-7 (FIG. 19), MDA-MB-231 (FIG. 20) and HT-29 (FIG. 21). The scale bar represents 100 μm. Over the same time period, there are no observable differences in proliferation rate for all cell-lines when comparing against their respective normal cultures. The morphology of the retrieved cells and their respective control experiments are also rather similar. This uniformity in cell behavior confirms that the retrieved cells were unaffected after isolation in the microdevice.

In clinical reports, a CTC count of 5 cancer cells per 7.5 ml of blood is significant to represent the severity of the disease (M. Cristofanilli, G. T. Budd, M. J. Ellis, A. Stopeck, J. Matera, M. C. Miller, J. M. Reuben, G. V. Doyle, W. J. Allard, L. W. Terstappen, D. F. Hayes, N. Engl. J. Med. 351, 781 (2004); F. Nole, E. Munzone, L. Zorzino, I. Minchella, M. Salvatici, E. Botteri, M. Medici, E. Verri, L. Adamoli, N. Rotmensz, A. Goldhirsch, M. T. Sandri, Ann. Oncol. 19, 891 (2008)).

In an experiment in accordance with an embodiment of the present invention, in order to test the effectiveness of the microdevice to function at this concentration, 1 or 2 cancer cells were manually picked out and added into 1 ml of PBS. The difficulties in getting reproducible cell numbers and the need for enough cells for the proliferation analysis restrict the characterization with such small cell numbers.

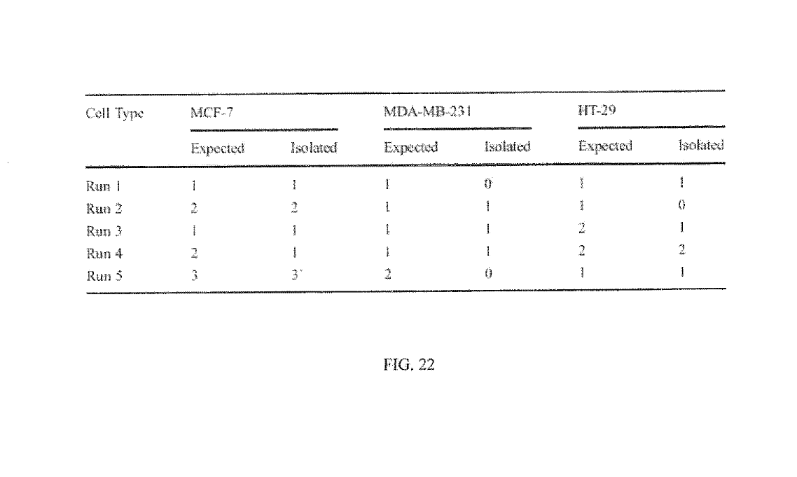

FIG. 22 is a table showing the number of cancer cells isolated from low concentrations of spiked sample solutions at a pressure input of 5 kPa, in accordance with an embodiment of the invention. The expected cell number refers to the number of cancer cells added to 1 ml of 1×PBS and isolated cell number refers to the number of cancer cells trapped. Out of the 15 experimental runs, 11 trials achieved the isolation and recovery of the spiked cancer cells with a pressure input of 5 kPa, which constitutes an 80% efficacy. This is in line with the results of the cancer cell isolation efficiency of the microdevice, which predicts an isolation efficiency of approximately 80%. Overall, a total of 16 cells from all 15 experimental runs were recovered.

A microfluidic platform in accordance with an embodiment of the invention has successfully demonstrated the enumeration of cancer cells of breast and colon origin in blood samples by utilizing cancer cells' inherently lower cell deformability and larger size as compared to blood constituents. This has potential in CTCs applications that can help to monitor the disease progression and treatment efficacy in contrast with biochemical techniques that are usually employed. The microdevice also achieves significant cancer cell isolation purity while preserving the integrity and state of these cells. With isolation efficiency of at least 80% for MCF-7, MDA-MB231 and HT-29, the microdevice can provide a potential useful assessment of the disease status. High cancer cell isolation purity for the microdevice will also minimize false positive results. The microdevice requires neither functional modification nor complex enrichment procedures, simplifying operation procedures. For clinical blood tests which usually handle larger sample volumes, parallel setups using multiple microdevices simultaneously can be adopted. Flowing 5 ml of sample solution through 3 microdevices at the same time at 5 kPa took no more than 2.5 hours which is considerably shorter than most leading techniques. These unique features make a microdevice in accordance with an embodiment of the invention attractive for possible CTC studies and potential clinical monitoring of the disease cancer.

In accordance with a further embodiment of the invention, a microdevice similar to those described above may be used to isolate cells to diagnose and/or monitor a disease or condition of a patient. In particular, a method may include diagnosing cancer, monitoring progression of cancer, monitoring treatment of cancer and/or providing a prognosis of a cancer in a patient. The diagnosis, monitoring or prognosis may be based on the isolated cells alone or made based on the isolated cells in conjunction with other tests. The isolated circulating tumor cells may be retrieved from the microfluidic device, or may be identified in situ within the microfluidic device. Where the microdevice is used in monitoring treatment, it may be used, for example, to monitor a patient's response to chemotherapy, drug treatment or other therapy. For example, if the number of CTCs isolated by the microdevice decreases (increases, or remains constant), this may reflect positively (or negatively) on the efficacy of the treatment.

In accordance with an embodiment of the invention, it will be appreciated that although isolation of cells from cell lines are referred to herein, a microdevice similar to those described above may be used to isolate cells from actual clinical samples. Further, a microdevice similar to those described above may be used to trap types of cells other than circulating tumor cells, including other diseased cells and non-cancer cells. For example, a microdevice similar to those described above may be used to isolate fetal cells, malaria infected cells, sickle anemia cells, dengue cells, stem cells and other types of cells from biological fluids, such as blood. When used with such non-cancer cells, the isolated cells may have a diameter of, for example, between about 6 μm and about 25 μm. It will be appreciated that other dimensions of such non-cancer cells may be isolated, depending on the size and type of cell to be isolated. Further, the microdevice may be used to isolate cells from biological fluids other than blood; for example, lymph, spinal fluid, urine, amniotic fluid or other biological fluids may be flowed through the device. In addition, the microdevice may be used to isolate cells from a separated component of whole blood, such as to isolate cells from a plasma component, a platelet component or a red blood cell component of blood. Further, the microdevice may be used to enrich samples.

Although transparent devices have been described herein, including those mounted on an inverted microscope, a microdevice in accordance with an embodiment of the invention may also be opaque, for example when mounted on silicon, in which case an upright microscope may be used instead of an inverted microscope.

As used herein, the term “biorheological property” means a property relating to the deformability and flow of a biological fluid or component thereof (such as a cell). A “biorheological property difference” means a difference between the biorheological properties of two or more biological fluids or components thereof (such as cells).

As used herein, where an isolation well comprises a cell trap of a size and shape suitable to mechanically isolate a cell within the cell trap, it should be appreciated that the size and shape of the cell trap may be determined by a variety of different possible techniques such as those described herein, based on the type of cell to be isolated. For example, the size and shape may be determined using experiments and/or computational fluid dynamics simulations, such as those provided above. As a result of such experiments, it may be determined that the cell trap is appropriately sized and shaped to isolate mostly single, or sometimes double, cells within a single cell trap. For instance, in the above experiments, a crescent, “U, “V” or “C” shaped cell trap with a longer dimension (the distance measured from the outermost edge of the tip of one “horn” of the crescent, “U,” “V” or “C” to the outermost edge of the tip of the other “horn” of the crescent, where outermost means the edge that is on the outer side of the crescent, which has a larger radius, as opposed to the inner bowl of the crescent, which has a smaller radius) of between about 15 μm to about 40 μm and a left or right tilt was determined to be of a size and shape suitable to isolate breast cancer cells. (For instance, in FIG. 5, the longer dimension is measured from the left edge of the tip of the left horn of isolation well 510 to the right edge of the tip of the right horn of isolation well 510). It will be appreciated that, based on the size, shape and other biorheological properties of the given cells to be isolated, other dimensions and shapes may be determined to be suitable in order to isolate the given cells. An inside radius of a “bowl” of a cell trap may be appropriately sized to isolate the given cells. Further, such experiments and simulations may be used to determine the size and shape of a gap that is suitable to prevent passage of the cells to be isolated but to permit passage of other components of the biological fluid through the cell trap. For example, in the above experiments, a gap of about 4-5 μm was determined to be of a suitable size and shape to prevent passage of breast cancer cells to be isolated, but to permit passage of blood cells and other components of the blood through the cell trap. It will be appreciated that any other appropriate technique may be used to determine sizes and shapes of cell traps and/or gaps, as long as a size and shape of cell trap and/or gap is achieved that is suitable to mechanically isolate the desired cells.

Further, in accordance with an embodiment of the invention, an isolation well need not be crescent-shaped. Instead, an isolation well may be any size or shape suitable to mechanically isolate a separate individual cell within the isolation well. For example, “U” shaped, “V” shaped or “C” shaped isolation wells may be used instead of (or in addition to) crescent-shaped isolation wells.

Further, in accordance with an embodiment of the invention, it should be noted that live cells need not be used; fixed cells may be used instead, or cells that are in any state of health or viability. Further, pressure ranges and other numerical ranges provided herein should not be taken as limiting. For example, pressure ranges may be altered; if fixed cells are used rather than live cells, the pressure range may be higher because fixed cells are more sturdy than live cells. Further, it will be appreciated that dimensions given herein should not be taken as limiting, since the dimensions used are dependent on the size and type of cell to be isolated. For example, in one embodiment, cells (such as circulating tumor cells) of a diameter between about 5 μm to about 40 μm may be isolated, depending on the size and type of cell that is the target of the device. As used herein, it should be appreciated that a “diameter” of a cell may refer to a cell that is elongated or otherwise not spherical in shape, in which case “diameter” is intended to refer to the longest dimension of the cell. In clinical samples, for example, renal cancer cells may have sizes of about 5 μm or 6 μm to about 15 μm, cells of melanoma of the skin may have sizes of about 22 μm to about 28 μm, cells of a malignant synovioma may have sizes of about 21 μm to about 26 μm, cells of carcinoma of the bronchus may have sizes of about 13 μm to about 19 μm or larger, such as 25 μm to 30 μm; cells of carcinoma of the stomach may have sizes of about 30 μm to about 40 μm (R. F. Alexander and A. I. Spriggs, J. Clin. Path. 13, 414 (1960)). However, dimensions of clinical samples may vary widely, and it will be appreciated that other ranges of dimensions may be found for these and other cell types. Cell lines may have different dimensions than clinical samples. Any given fabricated device may be designed to isolate cells within a small range of sizes, for example varying by as little as 1-2 μm from a desired target cell size for the device; or may be designed to isolate cells over a larger range of sizes. Given the variation in dimensions of clinical samples, it will be appreciated that various different possible amounts of variation from a target cell size within a given fabricated device may be permitted. Further, more than one size of isolation well may be used within the same fabricated device. Further, it should be noted that renal cancer cells as small as close to 5 μm or 6 μm have been positively stained, which are smaller than a blood cell. However, because the cancer cells are stiffer than blood cells, they can still be isolated by a microdevice according to the invention. It should therefore be appreciated that other physical properties than size may be used to isolate cells, such as shear modulus, stiffness, size and/or deformability. Further, the cells to be isolated may be smaller than the blood cells or other components of a biological fluid in which the cells to be isolated are included.

Table 1 provides examples of cancer cell lines that have been tested, in accordance with an embodiment of the invention.

TABLE 1
Tested Cell Lines

| | | | | |
|---|---|---|---|---|
| No | Description | Cancer Type | Efficiency | Remarks |

Cell Lines

| | | | | |
|---|---|---|---|---|
| 1 | MCF-7 | Breast Adenocarcinoma | 81.5% | |
| 2 | MDA-MB-231 | Breast Adenocarcinoma | 80.9% | |
| 3 | HT-29 | Colorectal Adenocarcinoma | 85.4% | |
| 4 | AGS | Gastric Adenocarcinoma | 78.9% | |
| 5 | N87 | Gastric carcinoma | 80.7% | |
| 6 | HepG2 | Hepatocellular carcinoma | 87.4% | |
| 7 | Huh7 | Hepatocellular carcinoma | 81.1% | |
| 8 | Cal27 | Tongue squamous cell | 81.6% | Lower |
| | | carcinoma | | pressure |
| | | | | used |
| 9 | FADU | Pharynx squamous cell | 80.6% | |
| | | carcinoma | | |
| 10 | TOV 211D | Ovarian cancer cell line | 91.8% | |

In addition, clinical samples of non-small cell lung carcinoma (NSCLC), nasopharyngeal carcinoma (NPC) and renal cell carcinoma have been tested, in accordance with an embodiment of the invention. An efficiency of 95.9% was achieved with 79 blood samples for the renal cell carcinoma. It will be appreciated that other types of clinical samples may be used.

Further, although examples of certain types of cancer cells are given above, it should also be appreciated that other types of cancer cells may be isolated; for example, breast cancer cells, colorectal cancer cells, kidney cancer cells, lung cancer cells, gastric cancer cells, prostate cancer cells, ovarian cancer cells, squamous cell cancer cells, hepatocellular cancer cells, nasopharyngeal cancer cells and other types of cancer cells may be isolated.

In addition, in accordance with an embodiment of the invention, the microdevice may be used to allow enumeration of cells, for example by counting the number of cells that have been isolated in the microdevice. Further, a microdevice may be used to determine a cell type, for example by permitting staining of cells within the microdevice. Such stained cells may be viewed by a microscope. The microdevice may be mounted on a microscope slide. For example, cells may be stained to determine whether the cells are CTCs or another type of cell. Further, an embodiment according to the invention may permit real time visualization of the isolation process; and/or may permit real time enumeration of isolated cells. For example, an imaging system may be connected to the device, to capture images from the device, and/or may receive light from the device, in order to permit real time visualization of the isolation process and/or to permit real time enumeration of isolated cells. In one example, the imaging system may view and/or digitize the image obtained through a microscope when the device is mounted on a microscope slide. For instance, the imaging system may include a digitizer and/or camera coupled to the microscope and to a viewing monitor and computer processor. The imaging system may, for example, allow a user to view stained cells, or cells that have not been stained. It will be appreciated that the imaging system may include appropriate light sources, microscopes, cameras, lenses, reflectors, detectors, digitizers, computers, processor devices, monitors and/or other appropriate imaging or optical devices to permit real time visualization of isolation and/or to permit real time enumeration of isolated cells. In addition, the imaging system may be used for other imaging of the device.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A microfluidic device for isolating cells from a biological fluid, the device comprising:
an inlet receiving the biological fluid flowed into the device; and
at least one array of a plurality of isolation wells receiving the biological fluid from the inlet, at least one isolation well of the plurality of isolation wells comprising a cell trap of a size and shape suitable to mechanically isolate a cell within the cell trap, the cell trap comprising at least one gap of a size and shape suitable to prevent passage of the cells to be isolated but to permit passage of other components of the biological fluid through the cell trap.

2. A microfluidic device according to claim 1, wherein the biological fluid comprises blood, wherein the cells to be isolated comprise circulating tumor cells and wherein the other components comprise blood cells.

3. A microfluidic device according to claim 2, further comprising:
a cell collection point receiving isolated circulating tumor cells from the array of isolation wells; and
a waste outlet receiving waste blood cells from the isolation wells.

4. A microfluidic device according to claim 1, wherein the cell trap comprises a crescent-shaped structure.

5. A microfluidic device according to claim 1, wherein the cell trap comprises at least one of a “U” shaped structure, a “V” shaped structure and a “C” shaped structure.

6. A microfluidic device according to claim 1, wherein the at least one array of the plurality of isolation wells comprises a plurality of rows of isolation wells.

7. A microfluidic device according to claim 6, wherein the isolation wells of the plurality of rows of isolation wells are offset from each other.

8. A microfluidic device according to claim 7, wherein the isolation wells of the plurality of rows of isolation wells are offset from each other by about 25 μm.

9. A microfluidic device according to claim 2, wherein the at least one array of the plurality of isolation wells comprises at least one row of isolation wells, the isolation wells of the at least one row being spaced apart from each other by a distance sufficient to prevent clogging of the at least one array by blood cells.

10. A microfluidic device according to claim 9, wherein the isolation wells of the at least one row are spaced apart by about 50 μm.

11. A microfluidic device according to claim 1, configured to generate a pressure differential between the inlet and a waste outlet of the device within the range of physiological pressure differences found in circulating whole blood.

12. A microfluidic device according to claim 1, further comprising a pre-filter receiving the biological fluid from the inlet and flowing pre-filtered biological fluid to the at least one array, the pre-filter being linked to a waste outlet of the device.

13. A microfluidic device according to claim 12, wherein the pre-filter comprises filter gaps of about 20 μm.

14. A microfluidic device according to claim 1, wherein the device comprises at least two sections of arrays of a plurality of isolation wells, the at least two sections being separated by a flow passage to a cell collection point receiving isolated cells from at least one of the sections of arrays.

15. A microfluidic device according to claim 1, wherein the device comprises at least one section of arrays of a plurality of isolation wells.

16. A microfluidic device according to claim 1, wherein a pressure differential between the inlet and a waste outlet of the device produces flow of the biological fluid through the at least one array of isolation wells to isolate the cells to be isolated.

17. A microfluidic device according to claim 1, configured to generate a reversed pressure differential between the inlet and a waste outlet of the device to produce a reversed flow of fluid in the device that permits retrieval of isolated cells.

18. A microfluidic device according to claim 1, wherein the cell trap comprises an open flow side permitting a reversed flow of fluid to free an isolated cell from the trap.

19. A microfluidic device according to claim 1, wherein the cell trap comprises a left or right tilted orientation, the at least one array of the plurality of isolation wells comprises a plurality of rows of isolation wells, and the plurality of rows of isolation wells comprises alternating left and right tilted orientations of the cell traps in successive rows of the plurality of rows of isolation wells.

20. A microfluidic device according to claim 1, wherein the cell trap comprises at least three microstructures separated by at least two gaps between the at least three microstructures, the at least three microstructures forming a shape that includes a wider open side of the shape that is opposite a side of the shape in which the at least two gaps are situated.

21. A microfluidic device according to claim 1, wherein the device isolates the cells to be isolated from the biological fluid based solely on biorheological property differences between the cells to be isolated and the other components of the biological fluid.

22. A microfluidic device according to claim 1, wherein the device permits retrieval of viable isolated circulating tumor cells from a blood sample.

23. A microfluidic device according to claim 1, wherein the cells to be isolated comprise at least one of breast cancer cells, colorectal cancer cells, kidney cancer cells, lung cancer cells, gastric cancer cells, prostate cancer cells, ovarian cancer cells, squamous cell cancer cells, hepatocellular cancer cells and nasopharyngeal cancer cells.

24. A microfluidic device according to claim 1, configured to generate a pressure differential between the inlet and a waste outlet of the device within the range from about 5 kPa to about 15 kPa.

25. A microfluidic device according to claim 1, wherein each of the at least one gaps is about 4 μm to about 5 μm in width.

26. A microfluidic device according to claim 1, wherein the cell trap is of a size and shape suitable to mechanically isolate a cell of between about 5 μm and about 40 μm in diameter.

27. A microfluidic device according to claim 1, wherein the cell trap is crescent shaped and between about 15 μm and about 40 μm in its longer dimension, the longer dimension being a distance measured from the outermost edge of a tip of one horn of the crescent to the outermost edge of a tip of the other horn of the crescent, the outermost edges being the edges that are on an outer side of the crescent, which has a larger radius, as opposed to an inner bowl of the crescent, which has a smaller radius.

28. A microfluidic device according to claim 27, wherein the cell trap comprises at least two gaps of about 4 μm to about 5 μm width.

29. A microfluidic device according to claim 1, wherein the cells to be isolated comprise diseased cells.

30. A microfluidic device according to claim 1, wherein the cells to be isolated comprise at least one of fetal cells, malaria infected cells, sickle anemia cells, dengue cells and stem cells.

31. A microfluidic device according to claim 30, wherein the cells to be isolated comprise a diameter of between about 6 μm and about 25 μm.

32. A microfluidic device according to claim 1, wherein the device is mounted on a microscope slide.

33. A microfluidic device according to claim 32, wherein the device on the microscope slide is mounted on an inverted microscope.

34. A microfluidic device according to claim 32, wherein the device on the microscope slide is mounted on an upright microscope.

35. A microfluidic device according to claim 1, wherein the device permits an isolation efficiency of at least about 80% for the cells to be isolated.

36. A microfluidic device according to claim 35, wherein the cells to be isolated comprise circulating tumor cells.

37. A microfluidic device according to claim 1, wherein the device permits cell integrity of isolated cells to be preserved after isolation.

38. A microfluidic device according to claim 1, wherein the device permits retrieval of cancer cells at a prevalence on the order of about 1 cancer cell in about 1 ml of blood.

39. A microfluidic device according to claim 1, wherein no functional biochemical modification of the device or the cells to be isolated is necessary to maintain integrity of isolated cells.

40. A microfluidic device according to claim 1, wherein the device isolates cells based solely on physical properties of the cells to be isolated, the physical properties comprising at least one of shear modulus, stiffness, size and deformability.

41. A microfluidic device according to claim 1, wherein the cell trap is of a size and shape suitable to mechanically isolate most often a single cell within the cell trap.

42. A microfluidic device according to claim 1, wherein the device permits real time visualization of isolation of the cell.

43. A microfluidic device according to claim 42, further comprising an imaging system to capture images from the device to permit real time visualization of isolation of the cell.

44. A microfluidic device according to claim 1, wherein the device permits real time enumeration of isolated cells.

45. A microfluidic device according to claim 44, further comprising an imaging system to capture images from the device to permit real time enumeration of isolated cells.

46. A microfluidic device according to claim 1, wherein the device permits enumeration of isolated cells.

47. A method for isolating cells from a biological fluid, the method comprising:
flowing the biological fluid into an inlet of a microfluidic device;
flowing the biological fluid from the inlet through at least one array of a plurality of isolation wells to isolate the cells to be isolated from the biological fluid, the cells being isolated by at least one isolation well of the plurality of isolation wells comprising a cell trap of a size and shape suitable to mechanically isolate a cell within the cell trap; and
permitting components of the biological fluid, other than the cells to be isolated, to pass through the at least one array, the components being permitted to pass by at least one gap in the cell trap of a size and shape suitable to prevent passage of the cells to be isolated but to permit other components of the biological fluid to pass through the cell trap.

48. A method according to claim 47, wherein the biological fluid comprises blood, wherein the cells to be isolated comprise circulating tumor cells and wherein the other components comprise blood cells.

49. A method according to claim 48, further comprising:
collecting isolated circulating tumor cells from the array of isolation wells at a cell collection point of the device; and
passing waste blood cells from the isolation wells to a waste outlet of the device.

50. A method according to claim 47, wherein the cell trap comprises a crescent-shaped structure.

51. A method according to claim 47, wherein the cell trap comprises at least one of a “U” shaped structure, a “V” shaped structure and a “C” shaped structure.

52. A method according to claim 47, comprising passing the biological fluid through a plurality of rows of isolation wells.

53. A method according to claim 52, wherein the isolation wells of the plurality of rows of isolation wells are offset from each other.

54. A method according to claim 53, wherein the isolation wells of the plurality of rows of isolation wells are offset from each other by about 25 μm.

55. A method according to claim 48, comprising passing the biological fluid through at least one row of isolation wells, the isolation wells of the at least one row being spaced apart from each other by a distance sufficient to prevent clogging of the at least one array by whole blood cells.

56. A method according to claim 55, wherein the isolation wells of the at least one row are spaced apart by about 50 μm.

57. A method according to claim 47, comprising operating the inlet and a waste outlet of the device at a pressure differential that is within the range of physiological pressure differences found in circulating whole blood.

58. A method according to claim 47, further comprising pre-filtering the biological fluid received from the inlet, flowing the pre-filtered biological fluid to the at least one array, and flowing waste from the pre-filtering to a waste outlet of the device.

59. A method according to claim 58, wherein the pre-filtering comprises flowing the biological fluid through filter gaps of about 20 μm.

60. A method according to claim 47, comprising passing the biological fluid through at least two sections of arrays of a plurality of isolation wells, the at least two sections being separated by a flow passage to a cell collection point receiving isolated cells from at least one of the sections of arrays.

61. A method according to claim 47, comprising passing the biological fluid through at least one section of arrays of a plurality of isolation wells.

62. A method according to claim 47, comprising operating the inlet and a waste outlet of the device at a pressure differential that produces flow of the biological fluid through the at least one array of isolation wells to isolate the cells to be isolated.

63. A method according to claim 47, comprising reversing a pressure differential between the inlet and a waste outlet of the device to produce a reversed flow of fluid in the device that permits retrieval of isolated cells.

64. A method according to claim 47, comprising reversing flow of fluid through the device to free an isolated cell from an open flow side of the cell trap.

65. A method according to claim 47, wherein the cell trap comprises a left or right tilted orientation, the at least one array of the plurality of isolation wells comprises a plurality of rows of isolation wells, and the plurality of rows of isolation wells comprises alternating left and right tilted orientations of the cell traps in successive rows of the plurality of rows of isolation wells.

66. A method according to claim 47, wherein the cell trap comprises at least three microstructures separated by at least two gaps between the at least three microstructures, the at least three microstructures forming a shape including a wider open side of the shape that is opposite a side of the shape in which the at least two gaps are situated.

67. A method according to claim 47, comprising isolating the cells to be isolated from the biological fluid based solely on biorheological property differences between the cells to be isolated and the other components of the biological fluid.

68. A method according to claim 47, comprising retrieving viable isolated circulating tumor cells from a blood sample.

69. A method according to claim 47, comprising isolating viable circulating tumor cells comprising at least one of breast cancer cells, colorectal cancer cells, kidney cancer cells, lung cancer cells, gastric cancer cells, prostate cancer cells, ovarian cancer cells, squamous cell cancer cells, hepatocellular cancer cells and nasopharyngeal cancer cells.

70. A method according to claim 47, comprising operating a pressure differential between the inlet and a waste outlet of the device within the range from about 5 kPa to about 15 kPa.

71. A method according to claim 47, wherein each of the at least one gaps is about 4 μm to about 5 μm in width.

72. A method according to claim 47, wherein the cell trap is crescent shaped and between about 15 μm and about 40 μm in its longer dimension, the longer dimension being a distance measured from the outermost edge of a tip of one horn of the crescent to the outermost edge of a tip of the other horn of the crescent, the outermost edges being the edges that are on an outer side of the crescent, which has a larger radius, as opposed to an inner bowl of the crescent, which has a smaller radius.

73. A method according to claim 72, wherein the cell trap comprises at least two gaps of about 4 μm to about 5 μm width.

74. A method according to claim 47, further comprising using the isolated cells to perform at least one of diagnosing and monitoring of a disease or condition of an individual in need of the at least one of diagnosis and monitoring.

75. A method according to claim 74, wherein the isolated cells comprise diseased cells.

76. A method according to claim 74, wherein the isolated cells comprise at least one of fetal cells, malaria infected cells, sickle anemia cells, dengue cells and stem cells.

77. A method according to claim 76, wherein the cells to be isolated comprise a diameter of between about 6 μm and about 25 μm.

78. A method according to claim 47, further comprising performing an enumeration of the isolated cells.

79. A method according to claim 47, further comprising determining a cell type of the isolated cells.

80. A method according to claim 79, wherein determining the cell type comprises staining the isolated cells within the microfluidic device.

81. A method according to claim 80, further comprising viewing the stained isolated cells under a microscope.

82. A method according to claim 81, wherein the microfluidic device is mounted on a microscope slide.

83. A method according to claim 81, wherein the stained cells comprise circulating tumor cells.

84. A method according to claim 47, wherein the microfluidic device is mounted on a microscope slide on an inverted microscope.

85. A method according to claim 47, wherein the microfluidic device is mounted on a microscope slide on an upright microscope.

86. A method according to claim 47, comprising isolating the cells to be isolated with an isolation efficiency of at least about 80%.

87. A method according to claim 86, wherein the cells to be isolated comprise circulating tumor cells.

88. A method according to claim 47, comprising preserving cell integrity of the cells to be isolated after isolation.

89. A method according to claim 47, comprising retrieving cancer cells that have a prevalence on the order of about 1 cancer cell in about 1 ml of blood.

90. A method according to claim 47, comprising performing no functional biochemical modification of the device or cells to be isolated while maintaining integrity of isolated cells.

91. A method according to claim 47, comprising isolating cells based solely on physical properties of the cells to be isolated, the physical properties comprising at least one of shear modulus, stiffness, size and deformability.

92. A method according to claim 47, wherein the cell trap is of a size and shape suitable to mechanically isolate most often a single cell within the cell trap.

93. A method according to claim 47, further comprising permitting real time visualization of isolating the cells.

94. A method according to claim 93, further comprising capturing images from the device with an imaging system to permit real time visualization of isolating the cells.

95. A method according to claim 47, further comprising permitting real time enumeration of isolated cells.

96. A method according to claim 95, further comprising capturing images from the device with an imaging system to permit real time enumeration of isolated cells.

97. A method according to claim 47, further comprising obtaining a biological sample from an individual.

98. A method of diagnosing cancer in an individual in need of diagnosis thereof, the method comprising:
flowing blood from a sample of the blood of the patient into an inlet of a microfluidic device;
flowing the blood from the inlet through at least one array of a plurality of isolation wells to isolate circulating tumor cells from the blood, wherein if circulating tumor cells are present the circulating tumor cells are isolated by at least one isolation well of the plurality of isolation wells comprising a cell trap of a size and shape suitable to mechanically isolate a circulating tumor cell within the cell trap; and
permitting blood cells to pass through the at least one array, the blood cells being permitted to pass by at least one gap in the cell trap of a size and shape suitable to permit blood cells to pass through the cell trap but to prevent passage of the circulating tumor cells to be isolated;
wherein the individual may be diagnosed with cancer based at least in part on the isolated circulating tumor cells.

99. A method according to claim 98, further comprising retrieving the isolated circulating tumor cells from the microfluidic device.

100. A method according to claim 98, further comprising identifying the isolated circulating tumor cells when they are situated within the microfluidic device.

101. A method of monitoring progression of cancer in an individual in need of monitoring thereof, the method comprising:
flowing blood from a sample of the blood of the individual into an inlet of a microfluidic device;
flowing the blood from the inlet through at least one array of a plurality of isolation wells to isolate circulating tumor cells from the blood, the circulating tumor cells being isolated by at least one isolation well of the plurality of isolation wells comprising a cell trap of a size and shape suitable to mechanically isolate a circulating tumor cell within the cell trap; and
permitting blood cells to pass through the at least one array, the blood cells being permitted to pass by at least one gap in the cell trap of a size and shape suitable to permit blood cells to pass through the cell trap but to prevent passage of the circulating tumor cells to be isolated;
wherein progression of the cancer in the individual may be monitored based at least in part on the isolated circulating tumor cells.

102. A method according to claim 101, wherein the individual is undergoing treatment for the cancer.

103. A method according to claim 101, further comprising retrieving the isolated circulating tumor cells from the microfluidic device.

104. A method according to claim 101, further comprising identifying the isolated circulating tumor cells when they are situated within the microfluidic device.

105. A method of monitoring treatment of cancer in an individual in need of monitoring thereof, the method comprising:
flowing blood from a sample of the blood of the individual into an inlet of a microfluidic device;
flowing the blood from the inlet through at least one array of a plurality of isolation wells to isolate circulating tumor cells from the blood, the circulating tumor cells being isolated by at least one isolation well of the plurality of isolation wells comprising a cell trap of a size and shape suitable to mechanically isolate a circulating tumor cell within the cell trap; and
permitting blood cells to pass through the at least one array, the blood cells being permitted to pass by at least one gap in the cell trap of a size and shape suitable to permit blood cells to pass through the cell trap but to prevent passage of the circulating tumor cells to be isolated;
wherein treatment of the cancer in the individual may be monitored based at least in part on the isolated circulating tumor cells.

106. A method according to claim 105, further comprising retrieving the isolated circulating tumor cells from the microfluidic device.

107. A method according to claim 105, further comprising identifying the isolated circulating tumor cells when they are situated within the microfluidic device.

108. A method according to claim 105, further comprising determining the efficacy of the treatment based on the number of circulating tumor cells isolated by the microfluidic device.

109. A method of providing a prognosis of cancer in an individual in need of prognosis thereof, the method comprising:
flowing blood from a sample of the blood of the individual into an inlet of a microfluidic device;
flowing the blood from the inlet through at least one array of a plurality of isolation wells to isolate circulating tumor cells from the blood, the circulating tumor cells being isolated by at least one isolation well of the plurality of isolation wells comprising a cell trap of a size and shape suitable to mechanically isolate a circulating tumor cell within the cell trap; and
permitting blood cells to pass through the at least one array, the blood cells being permitted to pass by at least one gap in the cell trap of a size and shape suitable to permit blood cells to pass through the cell trap but to prevent passage of the circulating tumor cells to be isolated;
wherein prognosis of the cancer in the individual may be provided based at least in part on the isolated circulating tumor cells.

110. A method according to claim 109, further comprising retrieving the isolated circulating tumor cells from the microfluidic device.

111. A method according to claim 109, further comprising identifying the isolated circulating tumor cells when they are situated within the microfluidic device.

112. A microfluidic device for isolating circulating tumor cells from a biological fluid, the device comprising:
an inlet receiving the biological fluid flowed into the device;
at least one array of a plurality of cell traps receiving the biological fluid from the inlet, each cell trap of a size and shape suitable to mechanically isolate a circulating tumor cell within the cell trap, the cell trap comprising at least one gap of a size and shape suitable to prevent passage of the circulating tumor cell to be isolated but to permit passage of blood cells through the cell trap;
a cell collection point receiving isolated circulating tumor cells from the array of isolation wells; and
a waste outlet receiving waste blood cells from the isolation wells;
wherein a reversed pressure differential between the inlet and a waste outlet of the device produces a reversed flow of fluid in the device that permits retrieval of isolated cells in the cell collection point.

113. A microfluidic device for isolating circulating tumor cells from a biological fluid, the device comprising:
an inlet receiving the biological fluid flowed into the device; and
at least one array of a plurality of cell traps receiving the biological fluid from the inlet, each cell trap is crescent shaped and of a size suitable to mechanically isolate a circulating tumor cell within the cell trap, the cell trap comprising at least one gap of a size and shape suitable to prevent passage of the circulating tumor cell to be isolated but to permit passage of blood cells through the cell trap.

114. A microfluidic device for isolating circulating tumor cells from a biological fluid, the device comprising:
an inlet receiving the biological fluid flowed into the device; and
at least one array of a plurality of cell traps receiving the biological fluid from the inlet, each cell trap of a size and shape suitable to mechanically isolate a circulating tumor cell within the cell trap,
wherein the cell trap comprises at least three microstructures separated by at least two gaps between the at least three microstructures, the at least three microstructures forming a shape that includes a wider open side of the shape that is opposite a side of the shape in which the at least two gaps are situated, the at least two gaps of a size and shape suitable to prevent passage of the circulating tumor cell to be isolated but to permit passage of blood cells through the cell trap.
